SEQUENCE LISTING (25 sequences)

SEQ ID NO: 1 - protein, 5 aa, synthetic construct
  REGION 1..5: note = CDR-H1
NYWMN

SEQ ID NO: 2 - protein, 19 aa, synthetic construct
  REGION 1..19: note = CDR-H2
  VARIANT 8: note = X can be any amino acid except N
EIRLKSNXYTTHYAESVKG

SEQ ID NO: 3 - protein, 6 aa, synthetic construct
  REGION 1..6: note = CDR-H3
HYYFDY

SEQ ID NO: 4 - protein, 16 aa, synthetic construct
  REGION 1..16: note = CDR-L1
RSSKSLLHSNGITYFF

SEQ ID NO: 5 - protein, 7 aa, synthetic construct
  REGION 1..7: note = CDR-L2
QMSNLAS

SEQ ID NO: 6 - protein, 9 aa, synthetic construct
  REGION 1..9: note = CDR-L3
AQNLELPPT

SEQ ID NO: 7 - protein, 19 aa, synthetic construct
  REGION 1..19: note = CDR-H2
EIRLKSNQYTTHYAESVKG

SEQ ID NO: 8 - protein, 19 aa, synthetic construct
  REGION 1..19: note = CDR-H2
EIRLKSNNYTTHYAESVKG

SEQ ID NO: 9 - protein, 117 aa, synthetic construct
  REGION 1..117: note = heavy chain variable region
  VARIANT 57: note = X can be any amino acid except N
EVQLVESGGGLVQPGGSMRLSCVASGFPFSNYWMNWVRQAPGKGLEWVGEIRLKSNXYTTHYAESVKGRFTISRDDSKNSLYLQMNSLKTEDTAVYYCTRHYYFDYWGQGTLVTVSS

SEQ ID NO: 10 - protein, 117 aa, synthetic construct
  REGION 1..117: note = heavy chain variable region
EVQLVESGGGLVQPGGSMRLSCVASGFPFSNYWMNWVRQAPGKGLEWVGEIRLKSNQYTTHYAESVKGRFTISRDDSKNSLYLQMNSLKTEDTAVYYCTRHYYFDYWGQGTLVTVSS

SEQ ID NO: 11 - protein, 117 aa, synthetic construct
  REGION 1..117: note = heavy chain variable region
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSS

SEQ ID NO: 12 - protein, 113 aa, synthetic construct
  REGION 1..113: note = light chain variable region
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKR

SEQ ID NO: 13 - protein, 4 aa, synthetic construct
  REGION 1..4: note = epitope
PDTR

SEQ ID NO: 14 - protein, 4 aa, synthetic construct
  REGION 1..4: note = epitope
PESR

SEQ ID NO: 15 - protein, 447 aa, synthetic construct
  REGION 1..447: note = heavy chain
  VARIANT 57: note = X can be any amino acid except N
EVQLVESGGGLVQPGGSMRLSCVASGFPFSNYWMNWVRQAPGKGLEWVGEIRLKSNXYTTHYAESVKGRFTISRDDSKNSLYLQMNSLKTEDTAVYYCTRHYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 16 - protein, 219 aa, synthetic construct
  REGION 1..219: note = light chain
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 1401 bp, synthetic construct
  misc_feature 1..1401: note = heavy chain
  source 1..1401: mol_type = other DNA
atgaagcacctgtggttctttctgctgctggtggccgctcctagatgggtgctgtctgaagtgcagctggtggaatctggcggaggattggttcagcctggcggctccatgagactgtcttgtgtggcctctggcttccccttctccaactactggatgaactgggtccgacaggcccctggcaaaggactggaatgggtcggagagatccggctgaagtccaaccagtacaccacacactacgccgagtccgtgaagggcagattcaccatctctcgggacgactccaagaactccctgtacctgcagatgaacagcctgaaaaccgaggacaccgccgtgtactactgcacccggcactactacttcgactactggggccagggcaccctggtcacagtttcttccgcttccaccaagggacccagcgtgttccctctggctccttccagcaagtctacctctggcggaacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccaagcagggacgagctgaccaagaaccaggtgtccctgacctgcctcgtgaagggattctacccctccgatatcgccgtggaatgggagtctaatggccagcctgagaacaactacaagacaacccctcctgtgctggactccgacggctcattcttcctgtactccaagctgacagtggacaagtccagatggcagcagggcaacgtgttctcctgctccgtgatgcatgagggcctgcacaaccactacacccagaagtccctgtctctgagccccggcaaatga

SEQ ID NO: 18 - DNA, 720 bp, synthetic construct
  misc_feature 1..720: note = light chain
  source 1..720: mol_type = other DNA
atggttctgcagacacaggtgttcatctccctgctgctgtggatctctggcgcctacggcgacatcgtgatgacccagtctccactgagcaaccccgtgacacctggcgagcctgcctccatctcttgccggtcctctaagtctctgctgcactccaacggcatcacctactttttctggtatctgcagaagcccggccagtctcctcagctgctgatctaccagatgtccaacctggcctctggcgtgcccgatagattttccggctctggctctggcaccgacttcaccctgagaatctccagagtggaagccgaggacgtgggcgtgtactactgtgcccagaacctggaactgcctcctacctttggccagggcaccaaggtggaaatcaagcggacagtggccgctccttccgtgtttatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 19 - protein, 447 aa, synthetic construct
  REGION 1..447: note = heavy chain
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 20 - protein, 460 aa, synthetic construct
  REGION 1..460: note = heavy chain
  VARIANT 76: note = X can be any amino acid except N
MKHLWFFLLLVAAPRWVLSEVQLVESGGGLVQPGGSMRLSCVASGFPFSNYWMNWVRQAPGKGLEWVGEIRLKSNXYTTHYAESVKGRFTISRDDSKNSLYLQMNSLKTEDTAVYYCTRHYYFDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 21 - protein, 239 aa, synthetic construct
  REGION 1..239: note = light chain
MVLQTQVFISLLLWISGAYGDIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 22 - protein, 447 aa, synthetic construct
  REGION 1..447: note = heavy chain
EVQLVESGGGLVQPGGSMRLSCVASGFPFSNYWMNWVRQAPGKGLEWVGEIRLKSNQYTTHYAESVKGRFTISRDDSKNSLYLQMNSLKTEDTAVYYCTRHYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 23 - protein, 460 aa, synthetic construct
  REGION 1..460: note = heavy chain
MKHLWFFLLLVAAPRWVLSEVQLVESGGGLVQPGGSMRLSCVASGFPFSNYWMNWVRQAPGKGLEWVGEIRLKSNQYTTHYAESVKGRFTISRDDSKNSLYLQMNSLKTEDTAVYYCTRHYYFDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 24 - protein, 30 aa, synthetic construct
APPAHGVTSAPDTRPAPGSTAPPAHGVTSA

SEQ ID NO: 25 - protein, 30 aa, synthetic construct
APPAHGVTSAPESRPAPGSTAPPAHGVTSA

(19) United States
(12) Patent Application Publication
(10) Pub. No.: US 2025/0257149 A1
(43) Pub. Date: Aug. 14, 2025

(54) ANTI-MUC1 ANTIBODY

(71) Applicant: GLYCOTOPE GMBH, Berlin (DE)

(72) Inventors: Johanna GELLERT, Berlin (DE); Anke FLECHNER, Berlin (DE); Doreen WEIGELT, Berlin (DE); Antje DANIELCZYK, Berlin (DE)

(73) Assignee: GLYCOTOPE GMBH, Berlin (DE)

(21) Appl. No.: 19/053,293

(22) Filed: Feb. 13, 2025

Related U.S. Application Data
(63) Continuation of application No. 17/055,303, filed on Nov. 13, 2020, now Pat. No. 12,297,289.

(30) Foreign Application Priority Data
May 18, 2018 (EP) 18173253.8

(51) Int. Cl. C07K 16/30; A61K 31/4745; A61K 39/00; A61K 39/395; A61K 47/54; A61K 47/65; A61K 47/68; A61P 35/00; A61P 35/02

(52) U.S. Cl. CPC C07K 16/3092; A61K 31/4745; A61K 39/39558; A61K 47/545; A61K 47/65; A61K 47/68037; A61K 47/6851; A61P 35/00; A61P 35/02; A61K 2039/505; C07K 2317/14; C07K 2317/24; C07K 2317/41; C07K 2317/55; C07K 2317/565; C07K 2317/73; C07K 2317/76; C07K 2317/92

(57) ABSTRACT

The present invention pertains to novel antibodies directed against the cancer antigen MUC1. In particular, an antibody with improved antigen binding was obtained by deleting a glycosylation site in the CDR-H2 of a known anti-MUC1 antibody.

### CROSS-REFERENCE TO RELATED APPLICATIONS

The present application is a Continuation of U.S. patent application Ser. No. 17/055,303, filed on Nov. 13, 2020, which claims priority under 35 U.S.C. § 371 to International Patent Application No. PCT/EP2019/062756, filed May 17, 2019, which claims priority to and the benefit of European Patent Application No. 18173253.8, filed on May 18, 2018. The contents of these applications are hereby incorporated by reference in their entireties.

### SEQUENCE LISTING

The instant application contains a Sequence Listing which has been submitted in ASCII format via EFS-Web and is hereby incorporated by reference in its entirety. Said ASCII copy, created on May 18, 2018, is named 127273-0103_SL.txt and is 34 kb in size.

### FIELD OF THE INVENTION

The present invention pertains to the field of antibodies. A mutated anti-MUC1 antibody with increased antigen binding affinity is provided. In particular, a mutated version of the humanized antibody PankoMab is provided wherein asparagine 57 of the heavy chain variable region is substituted by another amino acid. Thereby, the glycosylation site in the CDR2 region is deleted and the antigen binding affinity is increased. In specific embodiments, the present invention is directed to the therapeutic and diagnostic use of this antibody and to methods of producing such antibodies.

### BACKGROUND OF THE INVENTION

Antibodies against tumor-associated antigens are widely used therapeutics against cancers. Today, many anti-cancer antibodies are approved for human therapy. Some of these antibodies act by blocking certain signaling pathways which are critical for survival or proliferation of specific cancer cells. Other anti-cancer antibodies activate the patient's immune response against the targeted cancer cells, for example by initiating antibody-dependent cellular cytotoxicity (ADCC) via natural killer cells. This mechanism is induced by binding of the antibody's Fc part to Fc receptors on the immune cells.

An interesting and important group of antibodies are those directed against mucin proteins. Mucins are a family of high molecular weight, heavily glycosylated proteins produced by many epithelial tissues in vertebrates. They can be subdivided into mucin proteins which are membrane-bound due to the presence of a hydrophobic membrane-spanning domain that favors retention in the plasma membrane, and mucins which are secreted onto mucosal surfaces or secreted to become a component of saliva. The human mucin protein family consists of many family members, including membrane bound MUC1.

Increased mucin production occurs in many adenocarcinomas, including cancer of the pancreas, lung, breast, ovary, colon, etc. Mucins are also overexpressed in lung diseases such as asthma, bronchitis, chronic obstructive pulmonary disease or cystic fibrosis. Two membrane mucins, MUC1 and MUC4 have been extensively studied in relation to their pathological implication in the disease process. Moreover, mucins are also being investigated for their potential as diagnostic markers. Several antibodies directed against mucin proteins (Clin. Cancer Res., 2011 Nov. 1; 17(21):6822-30, PLoS One, 2011 Jan. 14; 6(1):e15921), in particular MUC1, are known in the art. However, their therapeutic efficacy could still be improved.

In view of this, there is a need in the art to provide therapeutic anti-MUC1 antibodies with improved properties.

### SUMMARY OF THE INVENTION

The present inventors have found that deleting the glycosylation site in the heavy chain variable region of the anti-MUC1 antibody PankoMab did not abolish antigen binding, but rather unexpectedly increased the antigen affinity of the antibody. This was in particular surprising as the glycosylation site is located in the second complementarity-determining region of the heavy chain variable region (CDR-H2). The CDRs are those regions of an antibody which are directly involved in antigen binding and provide the contact to the epitope. Therefore, generally modifying the amino acids of a CDR is expected to be detrimental to the antigen binding affinity. The humanized PankoMab antibody additionally comprises a glycosylation site in CDR-H2, which carries a large carbohydrate structure. This carbohydrate structure is present directly at the binding interface to the antigen and hence, was considered to be involved in antigen binding. However, as demonstrated in the examples, the PankoMab variant (N54Q) wherein the glycosylation site is deleted by substituting the amino acid carrying the carbohydrate structure exhibits an increased antigen binding affinity.

Therefore, in a first aspect, the present invention is directed to an antibody capable of binding to MUC1, which comprises
i. a heavy chain variable region comprising the complementarity-determining regions (CDRs) CDR-H1 having the amino acid sequence of SEQ ID NO: 1, CDR-H2 having the amino acid sequence of SEQ ID NO: 2 and CDR-H3 having the amino acid sequence of SEQ ID NO: 3, and
ii. a light chain variable region comprising the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6.

In a second aspect, the present invention provides a nucleic acid encoding the antibody according to the invention. Furthermore, in a third aspect an expression cassette or vector comprising the nucleic acid according to the invention and a promoter operatively connected with said nucleic acid and, in a fourth aspect, a host cell comprising the nucleic acid or the expression cassette or vector according to the invention are provided.

In a fifth aspect, the present invention provides a conjugate comprising the antibody according to the invention conjugated to a further agent.

In a sixth aspect, the present invention is directed to a composition comprising the antibody according to the invention, the nucleic acid according to the invention, the expression cassette or vector according to the invention, the host cell according to the invention, or the conjugate according to the invention.

According to a seventh aspect, the invention provides the antibody, the nucleic acid, the expression cassette or vector, the host cell, the composition or the conjugate according to the invention for use in medicine, in particular in the treatment, prevention or diagnosis of cancer.

In an eighth aspect, the invention provides a method of increasing the MUC1 binding affinity of an antibody comprising
i. a heavy chain variable region comprising the complementarity-determining regions (CDRs) CDR-H1 having the amino acid sequence of SEQ ID NO: 1, CDR-H2 having the amino acid sequence of SEQ ID NO: 8 and CDR-H3 having the amino acid sequence of SEQ ID NO: 3, and
ii. a light chain variable region comprising the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6,
the method comprising the step of substituting the amino acid residue at position 8 of CDR-H2 with any amino acid residue except asparagine, resulting in CDR-H2 having the amino acid sequence of SEQ ID NO: 2.

In a ninth aspect, the invention provides a method of producing an antibody with increased MUC1 binding affinity, comprising
a) providing a nucleic acid coding for an antibody which comprises
i) a heavy chain variable region comprising the complementarity-determining regions (CDRs) CDR-H1 having the amino acid sequence of SEQ ID NO: 1, CDR-H2 having the amino acid sequence of SEQ ID NO: 8 and CDR-H3 having the amino acid sequence of SEQ ID NO: 3, and
ii) a light chain variable region comprising the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6;
b) introducing a mutation into said nucleic acid to produce a mutated nucleic acid, wherein the mutation is introduced in the codon coding for the amino acid residue at position 8 of CDR-H2 so that said codon codes for any amino acid residue except asparagine; and
c) producing the antibody with increased MUC1 binding affinity by expressing the mutated nucleic acid in a host cell.

In a tenth aspect, the present invention provides a method for treating cancer in a subject in need thereof comprising, administering to the subject with cancer a therapeutically effective amount of the antibody according to the invention, the nucleic acid according to the invention, the expression cassette or vector according to the invention, or the host cell according to the invention.

In a eleventh aspect, the present invention provides kits or devices comprising the antibody according to the invention, and associated methods that are useful in the diagnosis, detecting or monitoring of MUC1 associated disorders such as cancer.

Other objects, features, advantages and aspects of the present invention will become apparent to those skilled in the art from the following description and appended claims. It should be understood, however, that the following description, appended claims, and specific examples, which indicate preferred embodiments of the application, are given by way of illustration only. Various changes and modifications within the spirit and scope of the disclosed invention will become readily apparent to those skilled in the art from reading the following.

### Definitions

As used herein, the following expressions are generally intended to preferably have the meanings as set forth below, except to the extent that the context in which they are used indicates otherwise.

The expression “comprise”, as used herein, besides its literal meaning also includes and specifically refers to the expressions “consist essentially of” and “consist of”. Thus, the expression “comprise” refers to embodiments wherein the subject-matter which “comprises” specifically listed elements does not comprise further elements as well as embodiments wherein the subject-matter which “comprises” specifically listed elements may and/or indeed does encompass further elements. Likewise, the expression “have” is to be understood as the expression “comprise”, also including and specifically referring to the expressions “consist essentially of” and “consist of”. The term “consist essentially of”, where possible, in particular refers to embodiments wherein the subject-matter comprises 20% or less, in particular 15% or less, 10% or less or especially 5% or less further elements in addition to the specifically listed elements of which the subject-matter consists essentially of.

The term “antibody” in particular refers to a protein comprising at least two heavy chains and two light chains connected by disulfide bonds. Each heavy chain is comprised of a heavy chain variable region (V_{H}) and a heavy chain constant region (C_{H}). Each light chain is comprised of a light chain variable region (V_{L}) and a light chain constant region (C_{L}). The heavy chain-constant region comprises three or—in the case of antibodies of the IgM- or IgE-type—four heavy chain-constant domains (C_{H1}, C_{H2}, C_{H3} and C_{H4}) wherein the first constant domain C_{H1} is adjacent to the variable region and may be connected to the second constant domain C_{H2} by a hinge region. The light chain-constant region consists only of one constant domain. The variable regions can be further subdivided into regions of hypervariability, termed complementarity determining regions (CDRs), interspersed with regions that are more conserved, termed framework regions (FR), wherein each variable region comprises three CDRs and four FRs. The variable regions of the heavy and light chains contain a binding domain that interacts with an antigen. The heavy chain constant regions may be of any type such as γ-, δ-, α-, μ- or ε-type heavy chains. Preferably, the heavy chain of the antibody is a γ-chain. Furthermore, the light chain constant region may also be of any type such as κ- or λ-type light chains. Preferably, the light chain of the antibody is a κ-chain. The terms “γ- (δ-, α-, μ- or ε-) type heavy chain” and “κ- (λ-) type light chain” refer to antibody heavy chains or antibody light chains, respectively, which have constant region amino acid sequences derived from naturally occurring heavy or light chain constant region amino acid sequences, especially human heavy or light chain constant region amino acid sequences. In particular, the amino acid sequence of the constant domains of a γ-type (especially γ1-type) heavy chain is at least 95%, especially at least 98%, identical to the amino acid sequence of the constant domains of a human γ (especially the human γ1) antibody heavy chain. Furthermore, the amino acid sequence of the constant domain of a κ-type light chain is in particular at least 95%, especially at least 98%, identical to the amino acid sequence of the constant domain of the human κ antibody light chain. The constant regions of the antibodies may mediate the binding of the immunoglobulin to host tissues or factors, including various cells of the immune system (e.g., effector cells) and the first component (C1q) of the classical complement system. The antibody can be e.g. a humanized, human or chimeric antibody.

The antigen-binding portion of an antibody usually refers to full length or one or more fragments of an antibody that retains the ability to specifically bind to an antigen. It has been shown that the antigen-binding function of an antibody can be performed by fragments of a full-length antibody. Examples of binding fragments of an antibody include a Fab fragment, a monovalent fragment consisting of the V_{L}, V_{H}, C_{L} and C_{H1} domains; a F(ab)₂ fragment, a bivalent fragment comprising two Fab fragments, each of which binds to the same antigen, linked by a disulfide bridge at the hinge region; a Fd fragment consisting of the V_{H} and CH1 domains; a Fv fragment consisting of the V_{L} and V_{H} domains of a single arm of an antibody; and a dAb fragment, which consists of a V_{H} domain.

The “Fab part” of an antibody in particular refers to a part of the antibody comprising the heavy and light chain variable regions (V_{H} and V_{L}) and the first domains of the heavy and light chain constant regions (C_{H1} and C_{L}). In cases where the antibody does not comprise all of these regions, then the term “Fab part” only refers to those of the regions V_{H}, V_{L}, C_{H1} and C_{L} which are present in the antibody. Preferably, “Fab part” refers to that part of an antibody corresponding to the fragment obtained by digesting a natural antibody with papain which contains the antigen binding activity of the antibody. In particular, the Fab part of an antibody encompasses the antigen binding site or antigen binding ability thereof. Preferably, the Fab part comprises at least the V_{H} region of the antibody.

The “Fc part” of an antibody in particular refers to a part of the antibody comprising the heavy chain constant regions 2, 3 and—where applicable—4 (C_{H2}, C_{H3} and C_{H4}). In particular, the Fc part comprises two of each of these regions. In cases where the antibody does not comprise all of these regions, then the term “Fc part” only refers to those of the regions C_{H2}, C_{H3} and C_{H4} which are present in the antibody. Preferably, the Fc part comprises at least the C_{H2} region of the antibody. Preferably, “Fc part” refers to that part of an antibody corresponding to the fragment obtained by digesting a natural antibody with papain which does not contain the antigen binding activity of the antibody. In particular, the Fc part of an antibody is capable of binding to the Fc receptor and thus, e.g. comprises an Fc receptor binding site or an Fc receptor binding ability.

The terms “antibody” and “antibody construct”, as used herein, refer in certain embodiments to a population of antibodies or antibody constructs, respectively, of the same kind. In particular, all antibodies or antibody constructs of the population exhibit the features used for defining the antibody or antibody construct. In certain embodiments, all antibodies or antibody constructs in the population have the same amino acid sequence. Reference to a specific kind of antibody, such as an antibody capable of specifically binding to MUC1, in particular refers to a population of this kind of antibody.

The term “antibody” as used herein also includes fragments and derivatives of said antibody. A “fragment or derivative” of an antibody in particular is a protein or glycoprotein which is derived from said antibody and is capable of binding to the same antigen, in particular to the same epitope as the antibody. Thus, a fragment or derivative of an antibody herein generally refers to a functional fragment or derivative. In particularly preferred embodiments, the fragment or derivative of an antibody comprises a heavy chain variable region. It has been shown that the antigen-binding function of an antibody can be performed by fragments of a full-length antibody or derivatives thereof. Examples of fragments of an antibody include (i) Fab fragments, monovalent fragments consisting of the variable region and the first constant domain of each the heavy and the light chain; (ii) F(ab)₂ fragments, bivalent fragments comprising two Fab fragments linked by a disulfide bridge at the hinge region; (iii) Fd fragments consisting of the variable region and the first constant domain CH1 of the heavy chain; (iv) Fv fragments consisting of the heavy chain and light chain variable region of a single arm of an antibody; (v) scFv fragments, Fv fragments consisting of a single polypeptide chain; (vi) (Fv)₂ fragments consisting of two Fv fragments covalently linked together; (vii) a heavy chain variable domain; and (viii) multibodies consisting of a heavy chain variable region and a light chain variable region covalently linked together in such a manner that association of the heavy chain and light chain variable regions can only occur intermolecular but not intramolecular. Derivatives of an antibody in particular include antibodies which bind to or compete with the same antigen as the parent antibody, but which have a different amino acid sequence than the parent antibody from which it is derived. These antibody fragments and derivatives are obtained using conventional techniques known to those with skill in the art.

A target amino acid sequence is “derived” from or “corresponds” to a reference amino acid sequence if the target amino acid sequence shares a homology or identity over its entire length with a corresponding part of the reference amino acid sequence of at least 75%, more preferably at least 80%, at least 85%, at least 90%, at least 93%, at least 95%, at least 97%, at least 98% or at least 99%. The “corresponding part” means that, for example, framework region 1 of a heavy chain variable region (FRH1) of a target antibody corresponds to framework region 1 of the heavy chain variable region of the reference antibody. In particular embodiments, a target amino acid sequence which is “derived” from or “corresponds” to a reference amino acid sequence is 100% homologous, or in particular 100% identical, over its entire length with a corresponding part of the reference amino acid sequence. A “homology” or “identity” of an amino acid sequence or nucleotide sequence is preferably determined according to the invention over the entire length of the reference sequence or over the entire length of the corresponding part of the reference sequence which corresponds to the sequence which homology or identity is defined. An antibody derived from a parent antibody which is defined by one or more amino acid sequences, such as specific CDR sequences or specific variable region sequences, in particular is an antibody having amino acid sequences, such as CDR sequences or variable region sequences, which are at least 75%, preferably at least 80%, at least 85%, at least 90%, at least 93%, at least 95%, at least 97%, at least 98% or at least 99% homologous or identical, especially identical, to the respective amino acid sequences of the parent antibody. In certain embodiments, the antibody derived from (i.e. derivative of) a parent antibody comprises the same CDR sequences as the parent antibody, but differs in the remaining sequences of the variable regions.

The term “antibody” as used herein also refers to multivalent and multispecific antibodies, i.e. antibody constructs which have more than two binding sites each binding to the same epitope and antibody constructs which have one or more binding sites binding to a first epitope and one or more binding sites binding to a second epitope, and optionally even further binding sites binding to further epitopes.

“Specific binding” preferably means that an agent such as an antibody binds stronger to a target such as an epitope for which it is specific compared to the binding to another target. An agent binds stronger to a first target compared to a second target if it binds to the first target with a dissociation constant (K_{d}) which is lower than the dissociation constant for the second target. Preferably the dissociation constant for the target to which the agent binds specifically is more than 100-fold, 200-fold, 500-fold or more than 1000-fold lower than the dissociation constant for the target to which the agent does not bind specifically. Furthermore, the term “specific binding” in particular indicates a binding affinity between the binding partners with an affinity constant Kₐ of at least 10⁶ M⁻¹, preferably at least 10⁷ M⁻¹, more preferably at least 10⁸ M⁻¹. An antibody specific for a certain antigen in particular refers to an antibody which is capable of binding to said antigen with an affinity having a Kₐ of at least 10⁶ M⁻¹, preferably at least 10⁷ M⁻¹, more preferably at least 10⁸ M⁻¹. For example, the term “anti-MUC1 antibody” in particular refers to an antibody specifically binding MUC1 and preferably is capable of binding to MUC1 with an affinity having a Kₐ of at least 10⁶ M⁻¹, preferably at least 10⁷ M⁻¹, more preferably at least 10⁸ M⁻¹.

The term “MUC1” refers to the protein MUC1, also known as mucin-1, polymorphic epithelial mucin (PEM) or cancer antigen 15-3, in particular to human MUC1 (Accession No. P15941). MUC1 is a member of the mucin family and encodes a membrane bound, glycosylated phosphoprotein. MUC1 has a core protein mass of 120-225 kDa which increases to 250-500 kDa with glycosylation. It extends 200-500 nm beyond the surface of the cell. The protein is anchored to the apical surface of many epithelial cells by a transmembrane domain. The extracellular domain includes a 20 amino acid variable number tandem repeat (VNTR) domain, with the number of repeats varying from 20 to 120 in different individuals. These repeats are rich in serine, threonine and proline residues which permits heavy O-glycosylation. In certain embodiments, the term “MUC1” refers to tumor-associated MUC1 (“TA-MUC1”). TA-MUC1 is MUC1 present on cancer cells. This MUC1 differs from MUC1 present on non-cancer cells in its much higher expression level, its localization and its glycosylation. In particular, TA-MUC1 is present apolarly over the whole cell surface in cancer cells, while in non-cancer cells MUC1 has a strictly apical expression and hence, is not accessible for systemically administered antibodies. Furthermore, TA-MUC1 has an aberrant O-glycosylation which exposes new peptide epitopes on the MUC1 protein backbone and new carbohydrate tumor antigens such as the Thomsen-Friedenreich antigen alpha (TFα).

“TFα”, also called Thomsen-Friedenreich antigen alpha or Core-1, refers to the disaccharide Gal-β1,3-GalNAc which is O-glycosidically linked in an alpha-anomeric configuration to the hydroxy amino acids serine or threonine of proteins in carcinoma cells.

The term “sialic acid” in particular refers to any N- or O-substituted derivatives of neuraminic acid. It may refer to both 5-N-acetylneuraminic acid and 5-N-glycolylneuraminic acid, but preferably only refers to 5-N-acetylneuraminic acid. The sialic acid, in particular the 5-N-acetylneuraminic acid preferably is attached to a carbohydrate chain via a 2,3- or 2,6-linkage. Preferably, in the antibodies described herein both 2,3- as well as 2,6-coupled sialic acids are present.

A “relative amount of glycans” according to the invention refers to a specific percentage or percentage range of the glycans attached to the antibodies of an antibody preparation or in a composition comprising antibodies, respectively. In particular, the relative amount of glycans refers to a specific percentage or percentage range of all glycans comprised in the antibodies and thus, attached to the polypeptide chains of the antibodies in an antibody preparation or in a composition comprising antibodies. 100% of the glycans refers to all glycans attached to the antibodies of the antibody preparation or in a composition comprising antibodies, respectively. For example, a relative amount of glycans carrying bisecting GlcNAc of 10% refers to a composition comprising antibodies wherein 10% of all glycans comprised in the antibodies and thus, attached to the antibody polypeptide chains in said composition comprise a bisecting GlcNAc residue while 90% of all glycans comprised in the antibodies and thus, attached to the antibody polypeptide chains in said composition do not comprise a bisecting GlcNAc residue. The corresponding reference amount of glycans representing 100% may either be all glycan structures attached to the antibodies in the composition, or all N-glycans, i.e. all glycan structures attached to an asparagine residue of the antibodies in the composition, or all complex-type glycans. The reference group of glycan structures generally is explicitly indicated or directly derivable from the circumstances by the skilled person.

The term “N-glycosylation” refers to all glycans attached to asparagine residues of the polypeptide chain of a protein. These asparagine residues generally are part of N-glycosylation sites having the amino acid sequence Asn-Xaa-Ser/Thr, wherein Xaa may be any amino acid except for proline. Likewise, “N-glycans” are glycans attached to asparagine residues of a polypeptide chain. The terms “glycan”, “glycan structure”, “carbohydrate”, “carbohydrate chain” and “carbohydrate structure” are generally used synonymously herein. N-glycans generally have a common core structure consisting of two N-acetylglucosamine (GlcNAc) residues and three mannose residues, having the structure Manα1,6-(Manα1,3-)Manβ1,4-GlcNAcβ1,4-GlcNAcβ1-Asn with Asn being the asparagine residue of the polypeptide chain. N-glycans are subdivided into three different types, namely complex-type glycans, hybrid-type glycans and high mannose-type glycans.

The numbers given herein, in particular the relative amounts of a specific glycosylation property, are preferably to be understood as approximate numbers. In particular, the numbers preferably may be up to 10% higher and/or lower, in particular up to 9%, 8%, 7%, 6%, 5%, 4%, 3%, 2% or 1% higher and/or lower.

In a “conjugate” two or more compounds are linked together. In certain embodiments, at least some of the properties from each compound are retained in the conjugate. Linking may be achieved by a covalent or non-covalent bond. Preferably, the compounds of the conjugate are linked via a covalent bond. The different compounds of a conjugate may be directly bound to each other via one or more covalent bonds between atoms of the compounds. Alternatively, the compounds may be bound to each other via a chemical moiety such as a linker molecule wherein the linker is covalently attached to atoms of the compounds. If the conjugate is composed of more than two compounds, then these compounds may, for example, be linked in a chain conformation, one compound attached to the next compound, or several compounds each may be attached to one central compound.

The term “nucleic acid” includes single-stranded and double-stranded nucleic acids and ribonucleic acids as well as deoxyribonucleic acids. It may comprise naturally occurring as well as synthetic nucleotides and can be naturally or synthetically modified, for example by methylation, 5′- and/or 3′-capping.

The term “expression cassette” in particular refers to a nucleic acid construct which is capable of enabling and regulating the expression of a coding nucleic acid sequence introduced therein. An expression cassette may comprise promoters, ribosome binding sites, enhancers and other control elements which regulate transcription of a gene or translation of an mRNA. The exact structure of expression cassette may vary as a function of the species or cell type, but generally comprises 5-untranscribed and 5′- and 3′-untranslated sequences which are involved in initiation of transcription and translation, respectively, such as TATA box, capping sequence, CAAT sequence, and the like. More specifically, 5-untranscribed expression control sequences comprise a promoter region which includes a promoter sequence for transcriptional control of the operatively connected nucleic acid. Expression cassettes may also comprise enhancer sequences or upstream activator sequences.

According to the invention, the term “promoter” refers to a nucleic acid sequence which is located upstream (5) of the nucleic acid sequence which is to be expressed and controls expression of the sequence by providing a recognition and binding site for RNA-polymerases. The “promoter” may include further recognition and binding sites for further factors which are involved in the regulation of transcription of a gene. A promoter may control the transcription of a prokaryotic or eukaryotic gene. Furthermore, a promoter may be “inducible”, i.e. initiate transcription in response to an inducing agent, or may be “constitutive” if transcription is not controlled by an inducing agent. A gene which is under the control of an inducible promoter is not expressed or only expressed to a small extent if an inducing agent is absent. In the presence of the inducing agent the gene is switched on or the level of transcription is increased. This is mediated, in general, by binding of a specific transcription factor.

The term “vector” is used here in its most general meaning and comprises any intermediary vehicle for a nucleic acid which enables said nucleic acid, for example, to be introduced into prokaryotic and/or eukaryotic cells and, where appropriate, to be integrated into a genome. Vectors of this kind are preferably replicated and/or expressed in the cells. Vectors comprise plasmids, phagemids, bacteriophages or viral genomes. The term “plasmid” as used herein generally relates to a construct of extrachromosomal genetic material, usually a circular DNA duplex, which can replicate independently of chromosomal DNA.

According to the invention, the term “host cell” relates to any cell which can be transformed or transfected with an exogenous nucleic acid. The term “host cells” comprises according to the invention prokaryotic (e.g. E. coli) or eukaryotic cells (e.g. mammalian cells, in particular human cells, yeast cells and insect cells). Particular preference is given to mammalian cells such as cells from humans, mice, hamsters, pigs, goats, or primates. The cells may be derived from a multiplicity of tissue types and comprise primary cells and cell lines. A nucleic acid may be present in the host cell in the form of a single copy or of two or more copies and, in one embodiment, is expressed in the host cell.

The term “patient” means according to the invention a human being, a nonhuman primate or another animal, in particular a mammal such as a cow, horse, pig, sheep, goat, dog, cat or a rodent such as a mouse and rat. In a particularly preferred embodiment, the patient is a human being.

The term “cancer” according to the invention in particular comprises leukemias, seminomas, melanomas, carcinomas, teratomas, lymphomas, sarcomas, mesotheliomas, neuroblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, bladder cancer, cancer of the uterus, ovarian cancer and lung cancer and the metastases thereof. The term cancer according to the invention also comprises cancer metastases.

By “tumor” is meant a group of cells or tissue that is formed by misregulated cellular proliferation. Tumors may show partial or complete lack of structural organization and functional coordination with the normal tissue, and usually form a distinct mass of tissue, which may be either benign or malignant.

By “metastasis” is meant the spread of cancer cells from its original site to another part of the body. The formation of metastasis is a very complex process and normally involves detachment of cancer cells from a primary tumor, entering the body circulation and settling down to grow within normal tissues elsewhere in the body. When tumor cells metastasize, the new tumor is called a secondary or metastatic tumor, and its cells normally resemble those in the original tumor. This means, for example, that, if breast cancer metastasizes to the lungs, the secondary tumor is made up of abnormal breast cells, not of abnormal lung cells. The tumor in the lung is then called metastatic breast cancer, not lung cancer.

The term “pharmaceutical composition” particularly refers to a composition suitable for administering to a human or animal, i.e., a composition containing components which are pharmaceutically acceptable. Preferably, a pharmaceutical composition comprises an active compound or a salt or prodrug thereof together with a carrier, diluent or pharmaceutical excipient such as buffer, preservative and tonicity modifier.

Numeric ranges described herein are inclusive of the numbers defining the range. The headings provided herein are not limitations of the various aspects or embodiments of this invention which can be read by reference to the specification as a whole. According to one embodiment, subject-matter described herein as comprising certain steps in the case of methods or as comprising certain ingredients in the case of compositions refers to subject-matter consisting of the respective steps or ingredients. It is preferred to select and combine preferred aspects and embodiments described herein and the specific subject-matter arising from a respective combination of preferred embodiments also belongs to the present disclosure.

### DETAILED DESCRIPTION OF THE INVENTION

The present invention is based on the development of a variant of the humanized anti-MUC1 antibody PankoMab wherein the glycosylation site in the CDR-H2 is deleted. Deletion of the glycosylation site was achieved by substituting amino acid Asn (asparagine) 57 of the heavy chain variable region by another amino acid, especially Gln (glutamine). Asn 57 is the acceptor amino acid residue of the glycosylation site to which the carbohydrate structure is attached. Substituting this asparagine residue by another residue abolishes glycosylation because the carbohydrate structure can only be transferred to an asparagine residue by the enzymes of the host cell. It was surprisingly found that deletion of the glycosylation site in the CDR-H2 of PankoMab increased the antigen binding affinity of the antibody.

In view of these findings, the present invention provides an antibody capable of binding to MUC1, which comprises
i) a heavy chain variable region comprising the complementarity-determining regions (CDRs) CDR-H1 having the amino acid sequence of SEQ ID NO: 1, CDR-H2 having the amino acid sequence of SEQ ID NO: 2 and CDR-H3 having the amino acid sequence of SEQ ID NO: 3, and
ii) a light chain variable region comprising the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6.

### Binding to MUC1

The antibody specifically binds to an epitope of MUC1. “Specific binding” means binding which is not non-specific adsorption. Examples of criteria for determination on whether binding is specific or not can include a dissociation constant (herein referred to as “K_{D}”). The epitope is in the extracellular tandem repeats of MUC1. In certain embodiments, the antibody binds to MUC1 in a glycosylation-dependent manner. In particular, the antibody binds stronger if said tandem repeats are glycosylated at a threonine residue with N-acetyl galactosamine (Tn), sialyl α2-6 N-acetyl galactosamine (sTn), galactose β1-3 N-acetyl galactosamine (TF) or galactose β1-3 (sialyl α2-6) N-acetyl galactosamine (sTF), preferably with Tn or TF. Preferably, the carbohydrate moiety is bound to the threonine residue by an α-O-glycosidic bond. The epitope in the tandem repeat domain of MUC1 in particular comprises the amino acid sequence PDTR (SEQ ID NO: 13) or PESR (SEQ ID NO: 14). The binding to this epitope preferably is glycosylation dependent, as described above, wherein in particular the binding is increased if the carbohydrate moiety described above is attached to the threonine residue of the sequence PDTR or PESR (SEQ ID NOs: 13 and 14), respectively.

The epitope is a tumor-associated MUC1 epitope (TA-MUC1). A TA-MUC1 epitope in particular refers to an epitope of MUC1 which is present on tumor cells but not on normal cells and/or which is only accessible by antibodies in the host's circulation when present on tumor cells but not when present on normal cells. In certain embodiments, the binding of the antibody to cells expressing TA-MUC1 epitope is stronger than the binding to cells expressing normal, non-tumor MUC1. Preferably, said binding is at least 1.5-fold stronger, preferably at least 2-fold stronger, at least 5-fold stronger, at least 10-fold stronger or at least 100-fold stronger. For TA-MUC1 binding, the antibody preferably specifically binds the glycosylated MUC1 tumor epitope such that the strength of the bond is increased at least by a factor 2, preferably a factor of 4 or a factor of 10, most preferably a factor of 20 in comparison with the bond to the non-glycosylated peptide of identical length and identical peptide sequence. Said binding can be assayed or determined by ELISA, RIA, surface plasmon resonance (hereinafter, referred to as “SPR”) analysis, or the like. Examples of equipment used in the SPR analysis can include BIAcore™ (manufactured by GE Healthcare Bio-Sciences Crop.), ProteOn™ (manufactured by Bio-Rad Laboratories, Inc.), DRX2 Biosensor (manufactured by Dynamic Biosensors GmbH), SPR-Navi™ (manufactured by BioNavis Oy Ltd.), Spreeta™ (manufactured by Texas Instruments Inc.), SPRi-Plexll™ (manufactured by Horiba, Ltd.), and Autolab SPR™ (manufactured by Metrohm). The binding of the antibody to the antigen expressed on cell surface can be assayed by flow cytometry or the like.

Furthermore, the antibody may exhibit antigen binding properties similar to those of a reference antibody comprising a heavy chain variable region with the amino acid sequence of SEQ ID NO: 11 or SEQ ID NO:10 and a light chain variable region with the amino acid sequence of SEQ ID NO: 12. Preferably, the reference antibody is the humanized antibody PankoMab. In particular, the antibody according to the invention specifically binds to the same antigen as the reference antibody, and preferably binds to said antigen with a higher affinity. That is, the antibody preferably binds to the antigen with an affinity having a dissociation constant which is lower than that of the reference antibody, more preferably at least 10% lower, at least 20% lower, at least 30% lower or at least 50% lower. Moreover, the antibody preferably shows cross-specificity with the reference antibody comprising a heavy chain variable region with the amino acid sequence of SEQ ID NO: 11 and a light chain variable region with the amino acid sequence of SEQ ID NO: 12. In particular, the humanized antibody is able to block the binding of the reference antibody to MUC1 if present in a high enough concentration. This is possible if the binding of the reference antibody to MUC1 is hindered when the antibody according to the invention is already bound to the antigen MUC1.

### The Anti-MUC1 Antibody

An antibody capable of binding to MUC1 of the present invention comprises a heavy chain variable region comprising the complementarity-determining regions (CDRs) CDR-H1 having the amino acid sequence of SEQ ID NO: 1, CDR-H2 having the amino acid sequence of SEQ ID NO: 2 and CDR-H3 having the amino acid sequence of SEQ ID NO: 3 and a light chain variable region comprising the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6.

In certain embodiments, the heavy chain variable region comprises an amino acid sequence which is at least 90% identical to the amino acid sequence of SEQ ID NO: 9. Especially, the heavy chain variable region comprises an amino acid sequence which is at least 95%, in particular at least 98% identical to the amino acid sequence of SEQ ID NO: 9. In these embodiments, the heavy chain variable region still comprises CDRs having the amino acid sequences of SEQ ID NOs: 1, 2 and 3. Hence, any sequence deviations to SEQ ID NO: 9 are located in the framework regions, but not in the CDRs. In particular, the heavy chain variable region comprises the amino acid sequence of SEQ ID NO: 9.

In certain embodiments, CDR-H2 has the amino acid sequence of SEQ ID NO: 2, wherein the amino acid at position 8 of SEQ ID NO: 2 is selected from the group consisting of glutamine, alanine, valine, histidine, tryptophan, tyrosine, lysine and arginine; especially glutamine, histidine, tryptophan, tyrosine, lysine and arginine. Preferably, the amino acid at position 8 of SEQ ID NO: 2 is glutamine, histidine, tryptophan, lysine or arginine, especially glutamine. In particular, CDR-H2 has the amino acid sequence of SEQ ID NO: 7.

In specific embodiments, the heavy chain variable region comprises an amino acid sequence which is at least 90% identical to the amino acid sequence of SEQ ID NO: 10. Especially, the heavy chain variable region comprises an amino acid sequence which is at least 95%, in particular at least 98% identical to the amino acid sequence of SEQ ID NO: 10. In these embodiments, the heavy chain variable region comprises the CDR-H1 having the amino acid sequence of SEQ ID NO: 1, CDR-H2 having the amino acid sequence of SEQ ID NO: 7 and CDR-H3 having the amino acid sequence of SEQ ID NO: 3. Hence, any sequence deviations to SEQ ID NO: 10 are located in the framework regions, but not in the CDRs. In particular, the heavy chain variable region comprises the amino acid sequence of SEQ ID NO: 10.

In certain embodiments, the light chain variable region comprises an amino acid sequence which is at least 90% identical to the amino acid sequence of SEQ ID NO: 12. Especially, the light chain variable region comprises an amino acid sequence which is at least 95%, in particular at least 98% identical to the amino acid sequence of SEQ ID NO: 12. In these embodiments, the light chain variable region still comprises CDRs having the amino acid sequences of SEQ ID NOs: 4, 5 and 6. Hence, any sequence deviations to SEQ ID NO: 12 are located in the framework regions, but not in the CDRs. In particular, the light chain variable region comprises the amino acid sequence of SEQ ID NO: 12.

In specific embodiments, the heavy chain variable region has an amino acid sequence which is at least 90% identical to the amino acid sequence of SEQ ID NO: 9, wherein the CDRs still have the amino acid sequences of SEQ ID NOs: 1, 2 and 3, and the light chain variable region has an amino acid sequence which is at least 90% identical to the amino acid sequence of SEQ ID NO: 12, wherein the CDRs still have the amino acid sequences of SEQ ID NOs: 4, 5 and 6. In particular, the heavy chain variable region has an amino acid sequence which is at least 95% identical to the amino acid sequence of SEQ ID NO: 9, wherein the CDRs still have the amino acid sequences of SEQ ID NOs: 1, 2 and 3, and the light chain variable region has an amino acid sequence which is at least 95% identical to the amino acid sequence of SEQ ID NO: 12, wherein the CDRs still have the amino acid sequences of SEQ ID NOs: 4, 5 and 6.

In specific embodiments, the heavy chain variable region has an amino acid sequence which is at least 90% identical to the amino acid sequence of SEQ ID NO: 10, wherein the CDRs still have the amino acid sequences of SEQ ID NOs: 1, 7 and 3, and the light chain variable region has an amino acid sequence which is at least 90% identical to the amino acid sequence of SEQ ID NO: 12, wherein the CDRs still have the amino acid sequences of SEQ ID NOs: 4, 5 and 6. In particular, the heavy chain variable region has an amino acid sequence which is at least 95% identical to the amino acid sequence of SEQ ID NO: 10, wherein the CDRs still have the amino acid sequences of SEQ ID NOs: 1, 7 and 3, and the light chain variable region has an amino acid sequence which is at least 95% identical to the amino acid sequence of SEQ ID NO: 12, wherein the CDRs still have the amino acid sequences of SEQ ID NOs: 4, 5 and 6.

In specific embodiments, the heavy chain variable region comprises an amino acid sequence which is at least 90% identical to the amino acid sequence represented by amino acid Nos 20 to 136 of SEQ ID NO: 20. Especially, the heavy chain variable region comprises an amino acid sequence which is at least 95%, in particular at least 98% identical to the amino acid sequence represented by amino acid Nos 20 to 136 of SEQ ID NO: 20. In these embodiments, the heavy chain variable region comprises the CDR-H1 having the amino acid sequence of SEQ ID NO: 1, CDR-H2 having the amino acid sequence of SEQ ID NO: 2 and CDR-H3 having the amino acid sequence of SEQ ID NO: 3. Hence, any sequence deviations to the amino acid sequence represented by amino acid Nos 20 to 136 of SEQ ID NO: 20 are located in the framework regions, but not in the CDRs. In particular, the heavy chain variable region comprises the amino acid sequence represented by amino acid Nos 20 to 136 of SEQ ID NO: 20. In certain embodiments, the amino acid at position 76 of SEQ ID NO: 20 is selected from the group consisting of glutamine, alanine, valine, histidine, tryptophan, tyrosine, lysine and arginine; especially glutamine, histidine, tryptophan, tyrosine, lysine and arginine. Preferably, the amino acid at position 76 of SEQ ID NO: 20 is glutamine, histidine, tryptophan, lysine or arginine, especially glutamine. In particular, CDR-H2 has the amino acid sequence of SEQ ID NO: 7 and/or the heavy chain variable region comprises the amino acid sequence represented by amino acid Nos 20 to 136 of SEQ ID NO: 23.

In specific embodiments, the light chain variable region comprises an amino acid sequence which is at least 90% identical to the amino acid sequence represented by amino acid Nos 21 to 133 of SEQ ID NO: 21. Especially, the light chain variable region comprises an amino acid sequence which is at least 95%, in particular at least 98% identical to the amino acid sequence represented by amino acid Nos 21 to 133 of SEQ ID NO: 21. In these embodiments, the light chain variable region still comprises CDRs having the amino acid sequences of SEQ ID NOs: 4, 5 and 6. Hence, any sequence deviations to amino acid sequence represented by amino acid Nos 21 to 133 of SEQ ID NO: 21 are located in the framework regions, but not in the CDRs. In particular, the light chain variable region comprises the amino acid sequence represented by amino acid Nos 21 to 133 of SEQ ID NO: 21.

In specific embodiments, the heavy chain variable region has an amino acid sequence which is at least 90% identical to the amino acid sequence represented by amino acid Nos 20 to 136 of SEQ ID NO: 20, wherein the CDRs still have the amino acid sequences of SEQ ID NOs: 1, 7 and 3, and the light chain variable region has an amino acid sequence which is at least 90% identical to the amino acid sequence represented by amino acid Nos 21 to 133 of SEQ ID NO: 21, wherein the CDRs still have the amino acid sequences of SEQ ID NOs: 4, 5 and 6. In particular, the heavy chain variable region has an amino acid sequence which is at least 95% identical to the amino acid sequence represented by amino acid Nos 20 to 136 of SEQ ID NO: 20, wherein the CDRs still have the amino acid sequences of SEQ ID NOs: 1, 7 and 3, and the light chain variable region has an amino acid sequence which is at least 95% identical to the amino acid sequence represented by amino acid Nos 21 to 133 of SEQ ID NO: 21, wherein the CDRs still have the amino acid sequences of SEQ ID NOs: 4, 5 and 6.

In specific embodiments, the heavy chain comprises an amino acid sequence which is at least 90% identical to the amino acid sequence of SEQ ID NO: 15. Especially, the heavy chain comprises an amino acid sequence which is at least 95%, in particular at least 98% identical to the amino acid sequence of SEQ ID NO: 15. In these embodiments, the heavy chain comprises the CDR-H1 having the amino acid sequence of SEQ ID NO: 1, CDR-H2 having the amino acid sequence of SEQ ID NO: 2 and CDR-H3 having the amino acid sequence of SEQ ID NO: 3. Hence, any sequence deviations to SEQ ID NO: 15 are located in the framework regions, but not in the CDRs. In particular, the heavy chain comprises the amino acid sequence of SEQ ID NO: 15. In certain embodiments, the amino acid at position 57 of SEQ ID NO: 15 is selected from the group consisting of glutamine, alanine, valine, histidine, tryptophan, tyrosine, lysine and arginine; especially glutamine, histidine, tryptophan, tyrosine, lysine and arginine. Preferably, the amino acid at position 57 of SEQ ID NO: 15 is glutamine, histidine, tryptophan, lysine or arginine, especially glutamine. In particular, CDR-H2 has the amino acid sequence of SEQ ID NO: 7 and/or the heavy chain variable region comprises the amino acid sequence represented by amino acid Nos 20 to 136 of SEQ ID NO: 22.

In specific embodiments, the light chain comprises an amino acid sequence which is at least 90% identical to the amino acid sequence of SEQ ID NO: 16. Especially, the light chain comprises an amino acid sequence which is at least 95%, in particular at least 98% identical to the amino acid sequence of SEQ ID NO: 16. In these embodiments, the light chain still comprises CDRs having the amino acid sequences of SEQ ID NOs: 4, 5 and 6. Hence, any sequence deviations to SEQ ID NO: 16 are located in the framework regions, but not in the CDRs. In particular, the light chain comprises the amino acid sequence of SEQ ID NO: 16.

In specific embodiments, the heavy chain has an amino acid sequence which is at least 90% identical to the amino acid sequence of SEQ ID NO: 15, wherein the CDRs still have the amino acid sequences of SEQ ID NOs: 1, 7 and 3, and the light chain variable region has an amino acid sequence which is at least 90% identical to the amino acid sequence of SEQ ID NO: 16, wherein the CDRs still have the amino acid sequences of SEQ ID NOs: 4, 5 and 6. In particular, the heavy chain has an amino acid sequence which is at least 95% identical to the amino acid sequence of SEQ ID NO: 15, wherein the CDRs still have the amino acid sequences of SEQ ID NOs: 1, 7 and 3, and the light chain has an amino acid sequence which is at least 95% identical to the amino acid sequence of SEQ ID NO: 16, wherein the CDRs still have the amino acid sequences of SEQ ID NOs: 4, 5 and 6.

The antibody according to the present invention includes and encompasses modified forms thereof. The modified form of the antibody of the present invention means an antibody of the present invention provided with chemical or biological modification. The chemically modified form includes forms wherein the amino acid skeleton is conjugated with a chemical moiety, forms having a chemically modified N-linked or O-linked carbohydrate chain, and the like. Said chemical moiety or form can be toxic or cytotoxic. Biologically modified forms include forms that have undergone post-translational modification (e.g., N-linked or O-linked glycosylation, N-terminal or C-terminal processing, deamidation, isomerization of aspartic acid, or oxidation of methionine), forms containing a methionine residue added to the N-terminus by expression using prokaryotic host cells, and the like. Such a modified form is also meant to include a form labeled to permit detection or isolation of the antibody of the present invention or the antigen thereof, for example, enzyme-labeled forms, fluorescently labeled forms, and affinity-labeled forms. Such a modified form of the antibody of the present invention is useful in improvement in the stability or blood retention of the original antibody of the present invention, reduction in antigenicity, detection or isolation of the antibody or the antigen, etc.

In particular, the antibody may comprise one or more modifications selected from the group consisting of defucosylation, reduced fucose, N-linked glycosylation, O-linked glycosylation, N-terminal processing, C-terminal processing, deamidation, isomerization of aspartic acid, oxidation of methionine, substitutions of two leucine (L) residues to alanine (A) at position 234 and 235 of the heavy chain (LALA), amidation of a proline residue and deletion or lack of one, two, or three amino acids at the carboxyl terminus. In specific embodiments, the antibody lacks one, two, or three carboxyl-terminal amino acid(s) at one or both heavy chains, or it lacks one carboxyl-terminal amino acid and the carboxyl-terminal proline residues is amidated at one or both heavy chains.

Such a modification may be made at an arbitrary position or the desired position in the antibody thereof. Alternatively, the same or two or more different modifications may be made at one or two or more positions therein.

For example, antibodies produced by cultured mammalian cells are known to lack a carboxyl-terminal lysine residue in its heavy chain (Journal of Chromatography A, 705: 129-134 (1995)). It is also known that occasionally 2 carboxyl-terminal amino acid residues (i.e., glycine and lysine) of a heavy chain are missing and that a proline residue newly located at the carboxyl terminus is amidated (Analytical Biochemistry, 360: 75-83 (2007)). Such lack or modification in these heavy chain sequences, however, affects neither the ability of the antibody to bind to its antigen nor the effector functions (complement activation, antibody-dependent cytotoxicity, etc.) of the antibody.

In certain embodiments, the antibody comprises a deletion or lack of 1 or 2 amino acid(s) in the carboxyl terminus of the heavy chain, and has an amidated residue (e.g., an amidated proline residue at the carboxyl-terminal site of the heavy chain). However, the antibody according to the present invention is not limited to the types described above as long as the deletion mutant maintains the ability to bind to the antigen.

In certain embodiments, two heavy chains of the antibody according to the present invention may be composed of any one type of heavy chain selected from the group consisting of the full length heavy chains and the heavy chains of the deletion mutant or may be composed of the combination of any two types selected therefrom. The quantitative ratio of the deletion variant heavy chain(s) depends on the type of cultured mammalian cells producing the antibody according to the present invention, and the culture conditions of the cells.

In specific embodiments, the antibody according to the present invention can include two heavy chains, both of which lack one carboxyl-terminal amino acid residue.

In specific embodiments, the antibody comprises the heavy chain having an amino acid sequence represented by amino acid Nos 1 to 446 of SEQ ID NO: 15 or 22 and the light chain having an amino acid sequence represented by amino acid Nos 1 to 219 of SEQ ID NO: 16. In certain embodiments, the amino acid at position 57 of SEQ ID NO: 15 is selected from the group consisting of glutamine, alanine, valine, histidine, tryptophan, tyrosine, lysine and arginine; especially glutamine, histidine, tryptophan, tyrosine, lysine and arginine. Preferably, the amino acid at position 57 of SEQ ID NO: 15 is glutamine, histidine, tryptophan, lysine or arginine, especially glutamine.

In certain embodiments, the antibody according to the present invention competes for the binding to TA-MUC1 with an antibody comprising a heavy chain variable region having the amino acid sequence of SEQ ID NO: 10 and a light chain variable region having the amino acid sequence of SEQ ID NO: 12, or an antibody comprising a heavy chain variable region having the amino acid sequence of SEQ ID NO: 11 and a light chain variable region having the amino acid sequence of SEQ ID NO: 12.

In certain embodiments, the antibody has the following properties: (a) specifically binding to MUC1, and/or (b) having the activity of being internalized into MUC1-expressing cells through binding to MUC1.

In certain embodiments, the antibody comprises at least one antibody heavy chain. Especially, the antibody comprises two antibody heavy chains. The antibody heavy chains in particular comprise a VH domain, a CH1 domain, a hinge region, a CH2 domain and a CH3 domain. In certain other embodiments, the antibody heavy chains comprise a CH2 domain and a CH3 domain, but do not comprise a CH1 domain. In further embodiments, one or more constant domains of the heavy chains may be replaced by other domains, in particular similar domains such as for example albumin. The antibody heavy chains may be of any type, including γ-, α-, ε-, δ- and μ-chains, and preferably are γ-chains, including γ1-, γ2-, γ3- and γ4-chains, especially γ1-chains. Hence, the antibody preferably is an IgG-type antibody such as an IgG1-, IgG3- or IgG4-type antibody, in particular an IgG1-type antibody.

In particular, the antibody further comprises at least one antibody light chain, especially two antibody light chains. The antibody light chains in particular comprise a VL domain and a CL domain. The antibody light chain may be a κ-chain or a λ-chain and especially is a κ-chain.

In certain embodiments, the antibody comprises two antibody heavy chains and two antibody light chains. In particular, the antibody comprises two antibody heavy chains of the γ1-type, each comprising a VH domain, a CH1 domain, a hinge region, a CH2 domain and a CH3 domain, and two antibody light chains of the κ-type, each comprising a VL domain and a CL domain.

In alternative embodiments, the antibody does not comprise an antibody light chain. In these embodiments, the light chain variable region may be fused to the N terminus of the heavy chain variable region or is inserted C terminal to the heavy chain variable region. Peptide linkers may be present to connect the light chain variable region with the remaining parts of the heavy chain.

In preferred embodiments, the antibody comprises an Fc region. The antibody may especially be a whole antibody, comprising two heavy chains each comprising the domains VH, CH1, hinge region, CH2 and CH3, and two light chains each comprising the domains VL and CL. The antibody in particular is capable of binding to one or more human Fcγ receptors, especially human Fcγ receptor IIIA. In alternative embodiments, the antibody does not or not significantly bind the human Fcγ receptor IIIA, and especially does not or not significantly bind to any human Fcγ receptor. In these embodiments the antibody in particular does not comprise a glycosylation site in the CH2 domain.

In alternative embodiments, the antibody does not comprise an Fc region. In these embodiments, the antibody in particular is a single chain variable region fragment (scFv) or another antibody fragment not comprising an Fc region.

### Glycosylation of the Anti-MUC1 Antibody

The anti-MUC1 antibody may comprise a CH2 domain in one or more antibody heavy chains. Natural human antibodies of the IgG type comprise an N-glycosylation site in the CH2 domain. The CH2 domains present in the antibody may or may not comprise an N-glycosylation site. In certain embodiments, the antibody does not comprise a glycosylation site in the CH2 domain. In particular, the antibody does not comprise an asparagine residue at the position in the heavy chain corresponding to position 297 according to the IMGT/Eu numbering system. For example, the antibody may comprise an Ala297 mutation in the heavy chain. In these embodiments, the antibody preferably has a strongly reduced ability or completely lacks the ability to induce, via binding to Fcγ receptors, antibody-dependent cellular cytotoxicity (ADCC) and/or antibody-dependent cellular phagocytosis (ADCP) and/or complement-dependent cytotoxicity (CDC). Strongly reduced ability in this respect in particular refers to a reduction to 10% or less, especially 3% or less, 1% or less or 0.1% or less activity compared to the same antibody comprising an N-glycosylation site in its CH2 domains and having a common mammalian glycosylation pattern such as those obtainable by production in human cell lines or in CHO cell lines, for example a glycosylation pattern as described herein. In these embodiments, the antibody in particular is an IgG1-type antibody.

In alternative embodiments, the CH2 domains present in the antibody comprise an N-glycosylation site. This glycosylation site in particular is at an amino acid position corresponding to amino acid position 297 of the heavy chain according to the IMGT/Eu numbering system and has the amino acid sequence motive Asn Xaa Ser/Thr wherein Xaa may be any amino acid except proline. The N-linked glycosylation at Asn297 is conserved in mammalian IgGs as well as in homologous regions of other antibody isotypes. Due to optional additional amino acids which may be present in the variable region or other sequence modifications, the actual position of this conserved glycosylation site may vary in the amino acid sequence of the antibody. Preferably, the glycans attached to the antibody are biantennary complex type N-linked carbohydrate structures, preferably comprising at least the following structure:

Asn-GlcNAc-GlcNAc-Man-(Man-GlcNAc)₂
wherein Asn is the asparagine residue of the polypeptide portion of the antibody; GlcNAc is N-acetylglucosamine and Man is mannose. The terminal GlcNAc residues may further carry a galactose residue, which optionally may carry a sialic acid residue. A further GlcNAc residue (named bisecting GlcNAc) may be attached to the Man nearest to the polypeptide. A fucose may be bound to the GlcNAc attached to the Asn. In these embodiments, the antibody in particular is an IgG1-type antibody.

In preferred embodiments, the antibody does not comprise N-glycolyl neuraminic acids (NeuGc) or detectable amounts of NeuGc. Furthermore, the antibody preferably also does not comprise Galili epitopes (Galα1,3-Gal structures) or detectable amounts of the Galili epitope. In particular, the relative amount of glycans carrying NeuGc and/or Galα1,3-Gal structures is less than 0.1% or even less than 0.02% of the total amount of glycans attached to the CH2 domains of the antibodies in the population of antibodies.

In particular, the antibody has a human glycosylation pattern. Due to these glycosylation properties, foreign immunogenic non-human structures which induce side effects are absent which means that unwanted side effects or disadvantages known to be caused by certain foreign sugar structures such as the immunogenic non-human sialic acids (NeuGc) or the Galili epitope (Gal-Gal structures), both known for rodent production systems, or other structures like immunogenic high-mannose structures as known from e.g. yeast systems are avoided.

In specific embodiments, the antibody comprises a glycosylation pattern having a detectable amount of glycans carrying a bisecting GlcNAc residue. In particular, the relative amount of glycans carrying a bisecting GlcNAc residue is at least 0.5%, especially at least 1% of the total amount of glycans attached to the glycosylation sites of the antibody in a composition. Furthermore, in certain embodiments the glycosylation pattern comprises a relative amount of glycans carrying at least one galactose residue of at least 25% of the total amount of glycans attached to the antibody in a composition. In particular, the relative amount of glycans carrying at least one galactose residue is at least 30%, especially at least 35% or at least 40% of the total amount of glycans attached to the antibody in a composition. In specific embodiments, the glycosylation pattern comprises a relative amount of glycans carrying at least one sialic acid residue of at least 1% of the total amount of glycans attached to the antibody in a composition. In particular, the relative amount of glycans carrying at least one sialic acid residue is at least 1.5%, especially at least 2% of the total amount of glycans attached to the antibody in a composition.

The antibody may have a glycosylation pattern having a high amount of core fucose or a low amount of core fucose. A reduced amount of fucosylation increases the ability of the antibody to induce ADCC. In certain embodiments, the relative amount of glycans carrying a core fucose residue is 40% or less, especially 30% or less or 20% or less of the total amount of glycans attached to the antibody in a composition. In alternative embodiments, the relative amount of glycans carrying a core fucose residue is at least 60%, especially at least 65% or at least 70% of the total amount of glycans attached to the antibody in a composition.

Via the presence or absence of the glycosylation site in the CH2 domain of the anti-MUC1 antibody and the presence or absence of fucose in the glycan structures at said glycosylation site, the ability of the antibody to induce ADCC and the strength of said ADCC induction can be controlled. The ADCC activity is increased by glycosylation of the Fc part of the antibody and further by reducing the amount of fucosylation in said glycosylation. In certain applications, fine tuning of the ADCC activity is important. Therefore, in certain situations, the antibody without a glycosylation site in the CH2 domain, the antibody with a glycosylation site in the CH2 domain and with a high amount of fucosylation, or the antibody with a glycosylation site in the CH2 domain and with a low amount of fucosylation may be most advantageous.

### Production of the Anti-MUC1 Antibody

The antibody is preferably recombinantly produced in a host cell. The host cell used for the production of the antibody may be any host cells which can be used for antibody production. Suitable host cells are in particular eukaryotic host cells, especially mammalian host cells. Exemplary host cells include yeast cells such as Pichia pastoris cell lines, insect cells such as SF9 and SF21 cell lines, plant cells, bird cells such as EB66 duck cell lines, rodent cells such as CHO, NS0, SP2/0 and YB2/0 cell lines, and human cells such as HEK293, PER.C6, CAP, CAP-T, AGE1.HN, Mutz-3 and KG1 cell lines.

In certain embodiments, the antibody is produced recombinantly in a human blood cell line, in particular in a human myeloid leukemia cell line. Preferred human cell lines which can be used for production of the antibody as well as suitable production procedures are described in WO 2008/028686 A2. In a specific embodiment, the antibody is obtained by expression in a human myeloid leukemia cell line selected from the group consisting of NM-H9D8, NM-H9D8-E6 and NM-H9D8-E6Q12 and cell lines derived therefrom. These cell lines were deposited under the accession numbers DSM ACC2806 (NM-H9D8; deposited on Sep. 15, 2006), DSM ACC2807 (NM-H9D8-E6; deposited on Oct. 5, 2006) and DSM ACC2856 (NM-H9D8-E6Q12; deposited on Aug. 8, 2007) according to the requirements of the Budapest Treaty at the Deutsche Sammlung von Mikroorganismen und Zellkulturen (DSMZ), InhoffenstraBe 7B, 38124 Braunschweig (DE) by Glycotope GmbH, Robert-Rössle-Str. 10, 13125 Berlin (DE). NM-H9D8 cells provide a glycosylation pattern with a high degree of sialylation, a high degree of bisecting GlycNAc, a high degree of galactosylation and a high degree of fucosylation. NM-H9D8-E6 and NM-H9D8-E6Q12 cells provide a glycosylation pattern similar to that of NM-H9D8 cells, except that the degree of fucosylation is very low. Other suitable cell lines include K562, a human myeloid leukemia cell line present in the American Type Culture Collection (ATCC CCL-243), as well as cell lines derived from the aforementioned.

In further embodiments, the antibody is produced recombinantly in CHO cells. Especially, the antibody may be produced recombinantly in a CHO dhfr− cell line such as the cell line of ATCC No. CRL-9096.

### Conjugates of the Anti-MUC1 Antibody

In certain embodiments, the antibody comprises one or more further agents conjugated thereto. The further agent may be any agent suitable for conjugation to the antibody. If more than one further agent is present in the antibody, these further agents may be identical or different, and in particular are all identical. Conjugation of the further agent to the antibody can be achieved using any methods known in the art. The further agent may be covalently, in particular by fusion or chemical coupling, or non-covalently attached to the antibody. In certain embodiments, the further agent is covalently attached to the antibody, especially via a linker moiety. The linker moiety may be any chemical entity suitable for attaching the further agent to the antibody.

The further agent preferably is useful in therapy, diagnosis, prognosis, detecting and/or monitoring of a disease, in particular cancer. For example, the further agent may be selected from the group consisting of radionuclides, chemotherapeutic agents, antibodies or antibody fragments, in particular those of different specificity than the anti-MUC1 antibody, e.g. checkpoint antibodies which block or activate immunomodulatory targets, enzymes, interaction domains, detectable labels, toxins, cytolytic components, immunomodulators, immunoeffectors, MHC class I or class II antigens, and liposomes. A particular preferred further agent is a radionuclide or a cytotoxic agent capable of killing cancer cells, such as a chemotherapeutic agent. In certain preferred embodiments, a chemotherapeutic agent is attached to the anti-MUC1 antibody forming a conjugate.

Specific examples of chemotherapeutic agents that can be conjugated as further agent include alkylating agents such as cisplatin, anti-metabolites, plant alkaloids and terpenoids, vinca alkaloids, podophyllotoxin, taxanes such as taxol, topoisomerase inhibitors such as irinotecan and topotecan, antineoplastics such as doxorubicin or microtubule inhibitors such as auristatins and maytansin/maytansinoids.

The chemotherapeutic agent may in particular be selected from a group consisting of a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an auristatin, a dolastatin, a maytansin, a maytansinoid, amatoxin, a methionine aminopeptidase, an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder, a DHFR inhibitor, an inhibitor of microtubule formation, a stabilizer of microtubule, a stabilizer of actin, a topoisomerase II inhibitor, a platinum compound, a ribosome inhibitor, an RNA polymerase II inhibitor and a bacterial toxin. In specific embodiments, the chemotherapeutic agent attached to the anti-MUC1 antibody is selected from the group consisting of an auristatin, a microtubule inhibitor such as maytansinoid, a DNA damaging agent, a DNA alkylating agent and a DNA minor groove binder.

In some embodiments of the chemotherapeutic agent is a maytansin or maytansinoid. Specific examples of maytansinoids useful for conjugation include maytansinol, N^{2′}-deacetyl-N^{2′}-(3-mercapto-1-oxopropyl)-maytansine (DM1), N^{2′}-deacetyl-N^{2′}-(4-mercapto-1-oxopentyl)-maytansine (DM3), and N^{2′}-deacetyl-N^{2′}-(4-methyl-4-mercapto-1-oxopentyl)-maytansine (DM4). In particular, DM1 or DM4 is attached to the anti-MUC1 antibody. In some embodiments, the chemotherapeutic agent attached to the anti-MUC1 antibody is an auristatin, in particular monomethyl auristatin F (MMAF), monomethyl auristatin E (MMAE) or auristatin T. In some embodiments, the chemotherapeutic agent attached to the anti-MUC1 antibody is a DNA minor groove binder, in particular pyrrolobenzodiazepine (PBD), pyrrolobenzodiazepine dimer (PBD dimer), duocarmycin, duocarmycin-hydroxybenzamide-azaindole (DUBA), seco-duocarmycin-hydroxybenzamide-azaindole (seco-DUBA) or doxorubicin. In some embodiments, the chemotherapeutic agent attached to the anti-MUC1 antibody is a DNA alkylating agent, in particular indolinobenzodiazepine or oxazolidinobenzodiazepine. In some embodiments, the chemotherapeutic agent attached to the anti-MUC1 antibody is a DNA damaging agent, in particular calicheamicin. In some embodiments, the chemotherapeutic agent attached to the anti-MUC1 antibody is an inhibitor of microtubule formation, in particular a tubulysin, an ansamitocin, podophyllotoxin or vinblastine. In some embodiments, the chemotherapeutic agent attached to the anti-MUC1 antibody is a stabilizer of microtubuli, in particular paclitaxel or an epothilone. In some embodiments, the chemotherapeutic agent attached to the anti-MUC1 antibody is a stabilizer of actin, in particular a phallotoxin. In some embodiments, the chemotherapeutic agent attached to the anti-MUC1 antibody is a topoisomerase II inhibitor, in particular teniposide, XK469, razoxane, amsacrine, idarubicin or mebarone. In some embodiments, the chemotherapeutic agent attached to the anti-MUC1 antibody is a platinum compound, in particular cisplatin, carboplatin, oxaliplatin, nedaplatin, triplatin tetranitrate, phenanthriplatin, picoplatin or sattraplatin. In some embodiments, the chemotherapeutic agent attached to the anti-MUC1 antibody is a ribosome inhibitor, in particular ricin, saporin, abrin, diphtheria toxin or exotoxin A. In some embodiments, the chemotherapeutic agent attached to the anti-MUC1 antibody is an RNA polymerase II inhibitor, in particular an amatoxin, such as, for example, amanitin. In some embodiments, the chemotherapeutic agent attached to the anti-MUC1 antibody is a bacterial toxin, in particular anthrax toxin. Suitable antibody drug conjugates are also described in EP 16 151 774.3 and LU 92659, to which is explicitly referred to herewith.

In certain embodiments, the further agent is a polypeptide or protein. This polypeptide or protein may in particular be fused to a polypeptide chain of the antibody. In certain embodiments, the further agent being a polypeptide or protein is fused to the C terminus of an antibody light chain of the antibody. In embodiments wherein the antibody comprises two antibody light chains, a further agent being a polypeptide or protein may be fused to the C terminus of each of the two antibody light chains. In further embodiments, the further agent being a polypeptide or protein is fused to the C terminus of an antibody heavy chain of the antibody. In embodiments wherein the antibody comprises two antibody heavy chains, a further agent being a polypeptide or protein may be fused to the C terminus of each of the two antibody heavy chains. The further agents may be identical or different and in particular have the same amino acid sequence. Suitable examples of such further agents being a polypeptide or protein may be selected from the group consisting of cytokines, chemokines, antibodies, antigen binding fragments, enzymes, and interaction domains.

In certain embodiments, the further agent being a polypeptide or protein is a checkpoint antibody which blocks and/or triggers activating signals. Examples of respective targets include CD40, CD3, CD137 (4-1BB), OX40, GITR, CD27, CD278 (ICOS), CD154 (CD40 ligand), CD270 (HVEM) and CD258 (LIGHT) as activating targets, CTLA4, PD1, CD80, CD244, A2AR, B7-H3 (CD276), B7-H4 (VTCN1), BTLA, IDO, KIR, LAG3, TIM-3, VISTA and phosphatidylserine as inhibitory targets, and their respective ligands such as PDL1. In specific examples, the anti-MUC1 antibody comprises two heavy chains and two light chains as described herein, wherein a scFv fragment specifically binding to CD3 is fused to the C terminus of each heavy chain; or wherein a scFv fragment specifically binding to PDL1 is fused to the C terminus of each light chain.

In further embodiments, the further agent being a polypeptide or protein is an immunomodulatory compound such as a chemokine, cytokine or growth factor. Suitable cytokines in this respect include interferons such as interferon-α, interferon-β and interferon-γ, and interleukins. Suitable growth factors include G-CSF and GM-CSF.

### The Nucleic Acid, Expression Cassette, Vector, Cell Line and Composition

In a further aspect, the present invention provides a nucleic acid encoding the antibody. The nucleic acid sequence of said nucleic acid may have any nucleotide sequence suitable for encoding the antibody. However, preferably the nucleic acid sequence is at least partially adapted to the specific codon usage of the host cell or organism in which the nucleic acid is to be expressed, in particular the human codon usage. The nucleic acid may be double-stranded or single-stranded DNA or RNA, preferably double-stranded DNA such as cDNA or single-stranded RNA such as mRNA. It may be one consecutive nucleic acid molecule or it may be composed of several nucleic acid molecules, each coding for a different part of the antibody. In preferred embodiments, the present invention provides a nucleotide sequence of the heavy chain of PankoMab variant (PM-N54Q) represented by SEQ ID NO: 17 and a nucleotide sequence of the light chain of PankoMab variant (PM-N54Q) represented by SEQ ID NO: 18.

If the antibody is composed of more than one different amino acid chain, such as a light chain and a heavy chain of the antibody, the nucleic acid may, for example, be a single nucleic acid molecule containing several coding regions each coding for one of the amino acid chains of the antibody, preferably separated by regulatory elements such as IRES elements in order to generate separate amino acid chains, or the nucleic acid may be composed of several nucleic acid molecules wherein each nucleic acid molecule comprises one or more coding regions each coding for one of the amino acid chains of the antibody. In addition to the coding regions encoding the antibody, the nucleic acid may also comprise further nucleic acid sequences or other modifications which, for example, may code for other proteins, may influence the transcription and/or translation of the coding region(s), may influence the stability or other physical or chemical properties of the nucleic acid, or may have no function at all.

In a further aspect, the present invention provides an expression cassette or vector comprising a nucleic acid according to the invention and a promoter operatively connected with said nucleic acid. In addition, the expression cassette or vector may comprise further elements, in particular elements which are capable of influencing and/or regulating the transcription and/or translation of the nucleic acid, the amplification and/or reproduction of the expression cassette or vector, the integration of the expression cassette or vector into the genome of a host cell, and/or the copy number of the expression cassette or vector in a host cell. Suitable expression cassettes and vectors comprising respective expression cassettes for expressing antibodies are well known in the prior art and thus, need no further description here.

Furthermore, the present invention provides a host cell comprising the nucleic acid according to the invention or the expression cassette or vector according to the invention. The host cell may be any host cell. It may be an isolated cell or a cell comprised in a tissue. Preferably, the host cell is a cultured cell, in particular a primary cell or a cell of an established cell line, preferably a tumor-derived cell. Preferably, it is a bacterial cell such as E. coli, a yeast cell such as a Saccharomyces cell, in particular S. cerevisiae, an insect cell such as a Sf9 cell, or a mammalian cell, in particular a human cell such as a tumor-derived human cell, a hamster cell such as CHO, or a primate cell. In a preferred embodiment of the invention the host cell is derived from human myeloid leukaemia cells. Preferably, it is selected from the following cells or cell lines: K562, KG1, MUTZ-3, or a cell or cell line derived therefrom, or a mixture of cells or cell lines comprising at least one of those aforementioned cells. The host cell is preferably selected from the group consisting of NM-H9D8, NM-H9D8-E6, NM H9D8-E6Q12, and a cell or cell line derived from anyone of said host cells. These cell lines and their properties are described in detail in the PCT-application WO 2008/028686 A2. In further embodiments, the host cell is of a CHO dhfr− cell line such as the cell line of ATCC No. CRL-9096. In preferred embodiments, the host cell is optimized for expression of glycoproteins, in particular antibodies, having a specific glycosylation pattern. Preferably, the codon usage in the coding region of the nucleic acid according to the invention and/or the promoter and the further elements of the expression cassette or vector are compatible with and, more preferably, optimized for the type of host cell used. Preferably, the antibody is produced by a host cell or cell line as described above.

In a specific aspect, the present invention provides a method of producing the antibody in a host cells as described herein. The method in particular comprises the steps of providing a host cell comprising a nucleic acid encoding the antibody, culturing the host cell under conditions suitable for expression of the antibody, and obtaining the antibody expressed by the host cell. The antibody according to the invention may be obtained or obtainable by said method.

In another aspect, the present invention provides a composition comprising the antibody, the nucleic acid, the expression cassette or vector, or the host cell. The composition may also contain more than one of these components. Furthermore, the composition may comprise one or more further components selected from the group consisting of solvents, diluents, and excipients Preferably, the composition is a pharmaceutical composition. In this embodiment, the components of the composition preferably are all pharmaceutically acceptable. The composition may be a solid or fluid composition, in particular a—preferably aqueous—solution, emulsion or suspension or a lyophilized powder.

### Use in Medicine

The antibody in particular is useful in medicine, in particular in therapy, diagnosis, prognosis, detecting and/or monitoring of a disease, in particular a disease as described herein, preferably cancer, infections, inflammatory diseases, graft-versus-host disease and immunodeficiencies.

Therefore, in a further aspect, the invention provides the antibody, the nucleic acid, the expression cassette or vector, the host cell, or the composition for use in medicine. Preferably, the use in medicine is a use in the treatment, prognosis, diagnosis, detecting and/or monitoring of a disease such as, for example, diseases associated with abnormal cell growth such as cancer, infections such as bacterial, viral, fungal or parasitic infections, inflammatory diseases such as autoimmune diseases and inflammatory bowel diseases, and diseases associated with a reduce immune activity such as immunodeficiencies. In a preferred embodiment, the disease is cancer.

Preferably, the cancer has a detectable expression of MUC1 or TA-MUC1, preferably detectable by immunohistochemistry, ELISA, RIA, enzyme-linked immunospot (ELISPOT) assay, dot blotting, Ouchterlony test or counter-immunoelectrophoresis (CIE), or in-situ hybridization. It especially includes cells having an MUC1 or TA-MUC1 expression which is detectable by immunohistochemistry or in-situ hybridization. The cancer may be tested on MUC1 or TA-MUC1 level prior to administration of the anti-MUC1 antibody.

The present invention further provides kits and devices comprising the antibody according to the invention, and associated methods that are useful in the diagnosis, detecting or monitoring of MUC1 associated disorders such as cancer. In some embodiments, a sandwich ELISA kit for testing, detecting or diagnosis comprising the antibody of the present invention is provided. This kit may further comprise one or more of a solution of MUC1 or TA-MUC1 protein standards, a coloring reagent, a buffer solution for dilution, an antibody for solid phase, an antibody for detection, and a washing solution, and the like. Preferably, the amount of the antibody bound to the antigen can be measured by the application of a method such as an absorbance, fluorescence, luminescence, or radioisotope (RI) method. Preferably, an absorbance plate reader, a fluorescence plate reader, a luminescence plate reader, an RI liquid scintillation counter, or the like is used in the measurement.

In some embodiments, the present invention provides the antibody according to the invention for use in immunohistochemistry (IHC) analysis, and a composition comprising the same.

The immunohistochemistry is not particularly limited as long as this approach involves reacting a tissue section with an antigen-binding antibody (primary antibody) and detecting the primary antibody bound with the antigen.

Different forms of cancers including metastases can be treated with the antibody according to the invention. The cancer can in particular be selected from the group consisting of colon cancer, lung cancer, ovarian cancer, breast cancer such as triple negative breast cancer, pancreatic cancer, cervical cancer, endometrial cancer, gastrointestinal cancer, kidney cancer, head and neck cancer, thyroid cancer and urothelial cancer. The cancer may further in particular be selected from stomach cancer, liver cancer, bladder cancer, skin cancer, prostate cancer and blood cancer. In certain embodiments, the cancer is a metastasizing cancer. The cancer may include any type of metastases, such as skin metastases, lymph node metastases, lung metastases, liver metastases, peritoneal metastases, pleural metastases and/or brain metastases. In certain embodiments, the cancer has an inflammatory phenotype. In these embodiments, any of the cancer types described above may be an inflammatory cancer.

In certain embodiments, the viral infection is caused by human immunodeficiency virus, herpes simplex virus, Epstein Barr virus, influenza virus, lymphocytic choriomeningitis virus, hepatitis B virus or hepatitis C virus. The inflammatory disease may be selected from inflammatory bowel disease, pelvic inflammatory disease, ischemic stroke, Alzheimer's disease, asthma, pemphigus vulgaris and dermatitis/eczema. The autoimmune disease may be selected from the group consisting of celiac disease, diabetes mellitus type 1, Graves' disease, inflammatory bowel disease, multiple sclerosis, psoriasis, rheumatoid arthritis, systemic lupus erythematosus, vitiligo, psoriatic arthritis, atopic dermatitis, scleroderma, sarcoidosis, primary biliary cirrhosis, Guillain-Barre syndrome, autoimmune hepatitis and ankylosing spondylitis. In certain embodiments, the disease comprises or is associated with cells which express MUC1, especially TA-MUC1. For example, a cancer to be treated is MUC1 positive, especially TA-MUC1 positive, i.e. comprises cancer cells which express MUC1, especially TA-MUC1.

In specific embodiments, the antibody is used for treatment in combination with another therapeutic agent, especially for treatment of cancer in combination with another anti-cancer agent. Said further therapeutic agent may be any known anti-cancer agent. Suitable anti-cancer therapeutic agents which may be combined with the antibody according to the invention may be chemotherapeutic agents, other antibodies, immunostimulatory agents, cytokines, chemokines, and vaccines. Furthermore, therapy with the antibody may be combined with radiation therapy, surgery and/or traditional Chinese medicine.

Anti-cancer agents that can be used in combination with the anti-MUC1 antibody may be selected from any chemotherapeutic agent, in particular chemotherapeutic agents known to be effective for treatment of MUC1 positive cancers. The type of chemotherapeutic agent also depends on the cancer to be treated. The combination partner may be selected from the group consisting of taxanes such as paclitaxel (Taxol), docetaxel (Taxotere) and SB-T-1214; cyclophosphamide; imatinib; pazopanib; capecitabine; cytarabine; vinorelbine; gemcitabine; anthracyclines such as daunorubicin, doxorubicin, epirubicin, idarubicin, valrubicin and mitoxantrone; aromatase inhibitors such as aminoglutethimide, testolactone (Teslac), anastrozole (Arimidex), letrozole (Femara), exemestane (Aromasin), vorozole (Rivizor), formestane (Lentaron), fadrozole (Afema), 4-hydroxyandrostenedione, 1,4,6-androstatrien-3,17-dione (ATD) and 4-androstene-3,6,17-trione (6-OXO); topoisomerase inhibitors such as irinotecan, topotecan, camptothecin, lamellarin D, etoposide (VP-16), teniposide, doxorubicin, daunorubicin, mitoxantrone, amsacrine, ellipticines, aurintricarboxylic acid and HU-331; platinum based chemotherapeutic agents such as cis-diamminedichloroplatinum(II) (cisplatin), cis-diammine(1,1-cyclobutanedicarboxylato)platinum(II) (carboplatin) and [(1R,2R)-cyclohexane-1,2-diamine](ethanedioato-O,O′)platinum(II) (oxaliplatin); PARP inhibitors such as olaparib, rucaparib and niraparib; TLR agonists such as imiquimod and resiquimod; and antimetabolites, in particular antifolates such as methotrexate, pemetrexed, raltitrexed and pralatrexate, pyrimidine analogues such as fluoruracil, gemcitabine, floxuridine, 5-fluorouracil and tegafur-uracil, and purine analogues, selective estrogen receptor modulators and estrogen receptor downregulators.

Furthermore, also therapeutic antibodies can be used as further combination partner. It may be any antibody that is useful in cancer therapy which is different from the anti-MUC1 antibody. In particular, the further antibody is approved for cancer treatment by an administration such as the U.S. Food and Drug Administration (FDA), the European Medicines Agency (EMA, formerly EMEA) and the Bundesinstitut für Arzneimittel und Medizinprodukte (BfArM). Examples of the further antibody that can be used for combination treatment are anti-EGFR antibodies such as Cetuximab, Tomuzotuximab, Panitumumab, Zalutumumab, Nimotuzumab, Matuzumab and Necitumumab; anti-HER2 antibodies such as Trastuzumab, Timigutuzumab and Pertuzumab; anti-VEGF antibodies such as bevacizumab (Avastin); anti-CD52 antibodies such as alemtuzumab (Campath); anti-CD30 antibodies such as brentuximab (Adcetris); anti-CD33 antibodies such as gemtuzumab (Mylotarg); and anti-CD20 antibodies such as rituximab (Rituxan, Mabthera), tositumomab (Bexxar) and ibritumomab (Zevalin). Further exemplary antibodies suitable for combination with the cancer therapy described herein include antibodies against antigens selected from the group consisting of Thomsen-Friedenreich antigen (TFα, TF3), Tn, Lewis Y, CD44, folate receptor a, NeuGc-GM3 ganglioside, DLL-3, RANKL, PTK7, Notch-3, Ephrin A4, insulin-like growth factor receptor 1, activin receptor-like kinase-1, claudin-6, disialoganglioside GD2, endoglin, transmembrane glycoprotein NMB, CD56, tumor-associated calcium signal transducer 2, tissue factor, ectonucleotide pyrophosphatase/phosphodiesterase 3, CD70, P-cadherin, mesothelin, six transmembrane epithelial antigen of the prostate 1 (STEAP1), carcinoembryonic antigen-related cell adhesion molecule 5 (CEACAM5), nectin 4, guanylyl cyclase C, solute carrier family 44 member 4 (SLC44A4), prostate-specific membrane antigen (PSMA), zinc transporter ZIP6 (LIV1 (ZIP6)), SLIT and NTRK-like protein 6 (SLITRK6), trophoblast glycoprotein (TPBG; 5T4), Fyn3, carbonic anhydrase 9, NaPi2b, fibronectin extra-domain B, endothelin receptor ETB, VEGFR2 (CD309), tenascin c, collagen IV and periostin.

The anti-MUC1 antibody can further be combined with checkpoint antibodies, i.e. antibodies blocking or activating immunomodulatory targets. Thereby, inhibitory signals for an immune response can be blocked and/or activating signals can be triggered. Examples of respective targets include CD40, CD3, CD137 (4-1BB), OX40, GITR, CD27, CD278 (ICOS), CD154 (CD40 ligand), CD270 (HVEM) and CD258 (LIGHT) as activating targets, CTLA4, PD1, CD80, CD244, A2AR, B7-H3 (CD276), B7-H4 (VTCN1), BTLA, IDO, KIR, LAG3, TIM-3, VISTA and phosphatidylserine as inhibitory targets, and their respective ligands such as PDL1.

In further embodiments, the anti-MUC1 antibody can be combined with the treatment with immunomodulatory compounds such as chemokines, cytokines, growth factors and vaccines. Suitable cytokines in this respect include interferons such as interferon-α, interferon-β and interferon-γ, and interleukins. Suitable growth factors include G-CSF and GM-CSF.

The anti-MUC1 antibody preferably is used for treatment of a primary tumor, a recurrent tumor and/or metastases of such tumors, and in particular is used for treatment before, during or after surgery and for the prevention or treatment of metastases. The anti-MUC1 antibody in particular is for the treatment of a patient as adjuvant therapy. In certain embodiments, the anti-MUC1 antibody is for the treatment of a patient as neoadjuvant therapy or in a combined neoadjuvant-adjuvant therapy. Furthermore, the anti-MUC1 antibody is for the treatment of a patient as palliative therapy.

The cancer therapy with the anti-MUC1 antibody preferably results in inhibition of tumor growth and in particular reduction of tumor size. Furthermore, the occurrence of further metastases is prevented and/or their number is reduced by the treatment. The treatment preferably results in an increase in progression-free survival; and/or an increase in lifespan and thus the overall survival.

The present invention further provides methods of therapy, diagnosis, prognosis, detecting and/or monitoring of a disease using the antibody according to the invention. The embodiments and examples of the use of the antibody in medicine also apply likewise to the medical methods. In particular, a method for treating a disease in a subject in need thereof comprising administering to the subject a therapeutically effective amount of the antibody according to the present invention is provided.

For example, the invention provides a method for treating cancer in a subject in need thereof comprising, administering to the subject with cancer a therapeutically effective amount of the antibody according to the invention. In specific embodiments, the cancer is characterized by expressing TA-MUC1. The cancer may be selected from the group consisting of ovarian cancer, breast cancer, pancreatic cancer, lung cancer, colon cancer, stomach cancer, liver cancer, kidney cancer, blood cancer, endometrial cancer, thyroid cancer, leukemia, seminomas, melanomas, carcinomas, teratomas, lymphomas, sarcomas, mesotheliomas, neuroblastomas, gliomas, rectal cancer, adrenal cancer, skin cancer, cancer of the brain, cervical cancer, intestinal cancer, intestine cancer, head and neck cancer, gastrointestinal cancer, lymph node cancer, esophagus cancer, colorectal cancer, ear, nose and throat (ENT) cancer, prostate cancer, bladder cancer, cancer of the uterus and the metastases thereof.

Furthermore, the invention provides a method for diagnosis, detecting or monitoring cancer, comprising the step of contacting a test sample with an antibody according to the invention.

### Methods of Increasing the MUC1 Binding Affinity

In a further aspect, the invention provides a method of increasing the MUC1 binding affinity of an antibody comprising
i) a heavy chain variable region comprising the complementarity-determining regions (CDRs) CDR-H1 having the amino acid sequence of SEQ ID NO: 1, CDR-H2 having the amino acid sequence of SEQ ID NO: 8 and CDR-H3 having the amino acid sequence of SEQ ID NO: 3, and
ii) a light chain variable region comprising the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6,
   the method comprising the step of substituting the amino acid residue at position 8 of CDR-H2 with any amino acid residue except asparagine, resulting in CDR-H2 having the amino acid sequence of SEQ ID NO: 2.

The antibody which MUC1 binding affinity is to be increased in particular is an antibody capable of binding to MUC1 as described herein, except that it comprises an asparagine at position 8 of the CDR-H2 sequence.

In certain embodiments, the heavy chain variable region of the antibody which MUC1 binding affinity is to be increased comprises an amino acid sequence which is at least 90% identical to the amino acid sequence of SEQ ID NO: 11. Especially, the heavy chain variable region comprises an amino acid sequence which is at least 95%, in particular at least 98% identical to the amino acid sequence of SEQ ID NO: 11. In these embodiments, the heavy chain variable region still comprises CDRs having the amino acid sequences of SEQ ID NOs: 1, 8 and 3. Hence, any sequence deviations to SEQ ID NO: 11 are located in the framework regions, but not in the CDRs. In particular, the heavy chain variable region comprises the amino acid sequence of SEQ ID NO: 11.

In certain embodiments, the light chain variable region of the antibody which MUC1 binding affinity is to be increased comprises an amino acid sequence which is at least 90% identical to the amino acid sequence of SEQ ID NO: 12. Especially, the light chain variable region comprises an amino acid sequence which is at least 95%, in particular at least 98% identical to the amino acid sequence of SEQ ID NO: 12. In these embodiments, the light chain variable region still comprises CDRs having the amino acid sequences of SEQ ID NOs: 4, 5 and 6. Hence, any sequence deviations to SEQ ID NO: 12 are located in the framework regions, but not in the CDRs. In particular, the light chain variable region comprises the amino acid sequence of SEQ ID NO: 12.

In specific embodiments, the heavy chain variable region of the antibody which MUC1 binding affinity is to be increased has an amino acid sequence which is at least 90% identical to the amino acid sequence of SEQ ID NO: 11, wherein the CDRs still have the amino acid sequences of SEQ ID NOs: 1, 8 and 3, and the light chain variable region has an amino acid sequence which is at least 90% identical to the amino acid sequence of SEQ ID NO: 12, wherein the CDRs still have the amino acid sequences of SEQ ID NOs: 4, 5 and 6. In particular, the heavy chain variable region has an amino acid sequence which is at least 95% identical to the amino acid sequence of SEQ ID NO: 11, wherein the CDRs still have the amino acid sequences of SEQ ID NOs: 1, 8 and 3, and the light chain variable region has an amino acid sequence which is at least 95% identical to the amino acid sequence of SEQ ID NO: 12, wherein the CDRs still have the amino acid sequences of SEQ ID NOs: 4, 5 and 6.

For example, the antibody which MUC1 binding affinity is to be increased is an anti-MUC1 antibody as disclosed in WO 2004/065423 A2 or WO 2011/012309 A1. In particular, the antibody which MUC1 binding affinity is to be increased is gatipotuzumab or PankoMab.

The antibody which MUC1 binding affinity is increased in particular is an antibody capable of binding to MUC1 as described herein.

In certain embodiments, MUC1 binding is as described herein. Increasing the MUC1 or TA-MUC1 binding affinity in particular refers to an increase of at least 10%, at least 20%, at least 33% or at least 50%. In preferred embodiments, MUC1 binding affinity is increased by at least 50%. The MUC1 binding affinity may be determined as described in the examples, especially using surface plasmon resonance analysis or switchSENSE® Technology (DRX2 Biosensor, manufactured by Dynamic Biosensors GmbH), as described, e.g., in example 4a and b.

In certain embodiments, the step of substituting the amino acid residue at position 8 of CDR-H2 is achieved by introducing a mutation into the nucleic acid coding for the antibody, wherein the mutation is introduced in the codon coding for said amino acid residue. Introducing the mutation can be done by any method. Several suitable methods are known in the art and the skilled person is capable of performing the necessary tasks to introduce the mutation. The antibody with increased MUC1 binding affinity can then be obtained by expressing the mutated nucleic acid, for example in a host cell. Nucleic acids, host cells and methods for producing the antibody are described herein and can be used for the method for increasing the MUC1 binding affinity.

In specific embodiments, the method of increasing the MUC1 binding affinity of an antibody comprises the steps of
a) providing a nucleic acid coding for the antibody which MUC1 binding affinity is to be increased
b) introducing a mutation into said nucleic acid to produce a mutated nucleic acid, wherein the mutation is introduced in the codon coding for the amino acid residue at position 8 of CDR-H2 so that said codon codes for any amino acid residue except asparagine; and
c) expressing the mutated nucleic acid to produce an antibody with increased MUC1 binding affinity.

The present invention further provides a method of producing an antibody with increased MUC1 binding affinity, comprising
a) providing a nucleic acid coding for an antibody which comprises
i) a heavy chain variable region comprising the complementarity-determining regions (CDRs) CDR-H1 having the amino acid sequence of SEQ ID NO: 1, CDR-H2 having the amino acid sequence of SEQ ID NO: 8 and CDR-H3 having the amino acid sequence of SEQ ID NO: 3, and
ii) a light chain variable region comprising the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6;
b) introducing a mutation into said nucleic acid to produce a mutated nucleic acid, wherein the mutation is introduced in the codon coding for the amino acid residue at position 8 of CDR-H2 so that said codon codes for any amino acid residue except asparagine; and
c) producing the antibody with increased MUC1 binding affinity by expressing the mutated nucleic acid in a host cell.

The embodiments, features and examples described herein for the other aspects, especially for the method of increasing the MUC1 binding affinity of an antibody, also likewise apply to the method of producing an antibody with increased MUC1 binding affinity.

In certain embodiments, the method of producing an antibody with increased MUC1 binding affinity further comprises a step (d) of processing the antibody with increased MUC1 binding affinity.

For example, processing the antibody with increased MUC1 binding affinity may include isolating the antibody from the cell culture. Isolation of the antibody in particular refers to the separation of the antibody from the remaining components of the cell culture. Separation of the antibody from the cell culture medium may be performed, for example, by chromatographic methods. Suitable methods and means for isolating antibodies are known in the art and can be readily applied by the skilled person.

The obtained antibody may optionally be subject to further processing steps such as e.g. modification steps such as chemical or enzymatic coupling of a further agent to the antibody, and/or formulation steps in order to produce the antibody in the desired quality and composition. Such further processing steps and methods are generally known in the art.

In further embodiments, step (d) additionally comprises the step of providing a pharmaceutical formulation comprising the antibody. Providing a pharmaceutical formulation comprising the antibody or formulating the antibody as a pharmaceutical composition in particular comprises exchanging the buffer solution or buffer solution components of the composition comprising the antibody. Furthermore, this step may include lyophilization of the antibody. In particular, the antibody is transferred into a composition only comprising pharmaceutically acceptable ingredients.

### SPECIFIC EMBODIMENTS

In the following, specific embodiments of the present invention are described.

Embodiment 1. An antibody capable of binding to MUC1, which comprises
i) a heavy chain variable region comprising the complementarity-determining regions (CDRs) CDR-H1 having the amino acid sequence of SEQ ID NO: 1, CDR-H2 having the amino acid sequence of SEQ ID NO: 2 and CDR-H3 having the amino acid sequence of SEQ ID NO: 3, and
ii) a light chain variable region comprising the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6.

Embodiment 2. The antibody according to Embodiment 1, wherein the amino acid at position 8 of the CDR-H2 is selected from the group consisting of glutamine, alanine, valine, histidine, tryptophan, tyrosine, lysine and arginine, especially glutamine, histidine, tryptophan, tyrosine, lysine and arginine, in particular glutamine.

Embodiment 3. The antibody according to Embodiment 1, wherein the amino acid at position 8 of the CDR-H2 is glutamine, histidine, arginine, tryptophan, or lysine.

Embodiment 4. The antibody according to Embodiments 1 to 3, wherein the CDR-H2 has the amino acid sequence of SEQ ID NO: 7.

Embodiment 6. An antibody capable of binding to MUC1, which comprises
i) a heavy chain variable region, which
a) has an amino acid sequence which is at least 90% identical to the amino acid sequence of SEQ ID NO: 9, and
b) comprises the complementarity-determining regions (CDRs) CDR-H1 having the amino acid sequence of SEQ ID NO: 1, CDR-H2 having the amino acid sequence of SEQ ID NO: 2 and CDR-H3 having the amino acid sequence of SEQ ID NO: 3, and
ii) a light chain variable region, which
a) has an amino acid sequence which is at least 90% identical to the amino acid sequence of SEQ ID NO: 12, and
b) comprises the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6.

Embodiment 7. An antibody capable of binding to MUC1, which comprises
i) a heavy chain variable region, which
a) has an amino acid sequence which is at least 95% identical to the amino acid sequence of SEQ ID NO: 9, and
b) comprises the complementarity-determining regions (CDRs) CDR-H1 having the amino acid sequence of SEQ ID NO: 1, CDR-H2 having the amino acid sequence of SEQ ID NO: 2 and CDR-H3 having the amino acid sequence of SEQ ID NO: 3, and
ii) a light chain variable region, which
a) has an amino acid sequence which is at least 95% identical to the amino acid sequence of SEQ ID NO: 12, and
b) comprises the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6.

Embodiment 8. The antibody according to Embodiment 6 or 7, wherein the amino acid at position 8 of the CDR-H2 is selected from the group consisting of glutamine, alanine, valine, histidine, tryptophan, tyrosine, lysine and arginine, especially glutamine, histidine, tryptophan, tyrosine, lysine and arginine, in particular glutamine.

Embodiment 9. The antibody according to Embodiment 7 or 8, wherein the amino acid at position 8 of the CDR-H2 is glutamine, histidine, arginine, tryptophan, or lysine.

Embodiment 10. An antibody capable of binding to MUC1, which comprises
i) a heavy chain variable region, which
   a) has an amino acid sequence which is at least 90% identical to the amino acid sequence of SEQ ID NO: 10, and
   b) comprises the complementarity-determining regions (CDRs) CDR-H1 having the amino acid sequence of SEQ ID NO: 1, CDR-H2 having the amino acid sequence of SEQ ID NO: 7 and CDR-H3 having the amino acid sequence of SEQ ID NO: 3, and
ii. a light chain variable region, which
   a) has an amino acid sequence which is at least 90% identical to the amino acid sequence of SEQ ID NO: 12, and
   b) comprises the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6.

Embodiment 11. An antibody capable of binding to MUC1, which comprises
i. a heavy chain variable region, which
   a) has an amino acid sequence which is at least 95% identical to the amino acid sequence of SEQ ID NO: 10, and
   b) comprises the complementarity-determining regions (CDRs) CDR-H1 having the amino acid sequence of SEQ ID NO: 1, CDR-H2 having the amino acid sequence of SEQ ID NO: 7 and CDR-H3 having the amino acid sequence of SEQ ID NO: 3, and
ii. a light chain variable region, which
   a) has an amino acid sequence which is at least 95% identical to the amino acid sequence of SEQ ID NO: 12, and
   b) comprises the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6.

Embodiment 12. An antibody capable of binding to MUC1, which comprises
i. a heavy chain variable region, which has the amino acid sequence of SEQ ID NO: 9, and
ii. a light chain variable region, which has the amino acid sequence of SEQ ID NO: 12.

Embodiment 13. The antibody according to Embodiment 12, wherein the amino acid at position 57 of SEQ ID NO: 9 is selected from the group consisting of glutamine, alanine, valine, histidine, tryptophan, tyrosine, lysine and arginine, especially glutamine, histidine, tryptophan, tyrosine, lysine and arginine, in particular glutamine.

Embodiment 14. The antibody according to Embodiment 12, wherein the amino acid at position 57 of SEQ ID NO: 9 is glutamine, histidine, arginine, tryptophan, or lysine.

Embodiment 15. An antibody capable of binding to MUC1, which comprises
i. a heavy chain variable region, which has the amino acid sequence of SEQ ID NO: 10, and
ii. a light chain variable region, which has the amino acid sequence of SEQ ID NO: 12.

Embodiment 16. An antibody capable of binding to MUC1, which comprises
i. a heavy chain variable region, which
   a) has an amino acid sequence which is at least 90% or at least 95% identical to the amino acid sequence represented by amino acid Nos 20 to 136 of SEQ ID NO: 20, and
   b) comprises the complementarity-determining regions (CDRs) CDR-H1 having the amino acid sequence of SEQ ID NO: 1, CDR-H2 having the amino acid sequence of SEQ ID NO: 2 or 7 and CDR-H3 having the amino acid sequence of SEQ ID NO: 3, and
ii. a light chain variable region, which
   a) has an amino acid sequence which is at least 90% or at least 95% identical to the amino acid sequence represented by amino acid Nos 21 to 133 of SEQ ID NO: 21, and
   b) comprises the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6.

Embodiment 17. An antibody capable of binding to MUC1, which comprises
i. a heavy chain variable region, which has the amino acid sequence represented by amino acid Nos 20 to 136 of SEQ ID NO: 20 or 23, and
ii. a light chain variable region, which has amino acid sequence represented by amino acid Nos 21 to 133 of SEQ ID NO: 21.

Embodiment 18. An antibody capable of binding to MUC1, which comprises
i. a heavy chain, which
   a) has an amino acid sequence which is at least 90% or at least 95% identical to the amino acid sequence of SEQ ID NO: 15, and
   b) comprises the complementarity-determining regions (CDRs) CDR-H1 having the amino acid sequence of SEQ ID NO: 1, CDR-H2 having the amino acid sequence of SEQ ID NO: 2 or 7 and CDR-H3 having the amino acid sequence of SEQ ID NO: 3, and
ii. a light chain variable region, which
   a) has an amino acid sequence which is at least 90% or 95% identical to the amino acid sequence of SEQ ID NO: 16, and
   b) comprises the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6.

Embodiment 19. An antibody capable of binding to MUC1, which comprises
i. a heavy chain variable region, which has the amino acid sequence of SEQ ID NO: 15 or SEQ ID NO: 22, and
ii. a light chain variable region, which has the amino acid sequence of SEQ ID NO: 16.

Embodiment 20. An antibody capable of binding to MUC1, which comprises
i. a heavy chain variable region, which
   a) has an amino acid sequence which is at least 90% or at least 95% identical to the amino acid sequence represented by amino acid Nos 20 to 460 of SEQ ID NO: 20, and
   b) comprises the complementarity-determining regions (CDRs) CDR-H1 having the amino acid sequence of SEQ ID NO: 1, CDR-H2 having the amino acid sequence of SEQ ID NO: 2 or 7 and CDR-H3 having the amino acid sequence of SEQ ID NO: 3, and
ii. a light chain variable region, which
   a) has an amino acid sequence which is at least 90% or 95% identical to the amino acid sequence represented by amino acid Nos 21 to 239 of SEQ ID NO: 21, and
   b) comprises the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6.

Embodiment 21. An antibody capable of binding to MUC1, which comprises
i. a heavy chain, which has the amino acid sequence represented by amino acid Nos 20 to 460 of SEQ ID NO: 20 or 23, and
ii. a light chain, which has amino acid sequence represented by amino acid Nos 21 to 239 of SEQ ID NO: 21.

Embodiment 22. The antibody according to any one of Embodiments 1 to 21, wherein the antibody comprises at least one heavy chain, comprising the heavy chain variable region, a CH1 domain, a hinge region, a CH2 domain and a CH3 domain.

Embodiment 23. The antibody according to any one of Embodiments 1 to 21, wherein the antibody comprises two heavy chains, each comprising the heavy chain variable region, a CH1 domain, a hinge region, a CH2 domain and a CH3 domain.

Embodiment 24. The antibody according to Embodiment 22 or 23, wherein the antibody is an IgG-type antibody, in particular an IgG1, IgG2 or IgG4-type antibody.

Embodiment 25. The antibody according to any one of Embodiments 1 to 24, wherein the antibody comprises at least one light chain, comprising the light chain variable region and a CL domain.

Embodiment 26. The antibody according to any one of Embodiments 1 to 24, wherein the antibody comprises two light chains, each comprising the light chain variable region and a CL domain.

Embodiment 274. The antibody according to Embodiment 25 or 26, wherein the light chain is a κ-type light chain.

Embodiment 28. The antibody according to any one of Embodiments 1 to 27, wherein the antibody does not comprise an N-glycosylation site in the CH2 domain.

Embodiment 29. The antibody according to any one of Embodiments 1 to 27, wherein the antibody comprises an N-glycosylation site in the CH2 domain of the antibody heavy chains.

Embodiment 30. The antibody according to Embodiment 29, wherein the antibody has a glycosylation pattern having one or more of the following characteristics
a relative amount of glycans carrying a bisecting GlcNAc residue of at least 0.5% of the total amount of glycans attached to the glycosylation sites of the antibody in a composition;
a relative amount of glycans carrying at least one galactose residue of at least 30% of the total amount of glycans attached to the glycosylation sites of the antibody in a composition;
a relative amount of glycans carrying a core fucose residue of at least 60% of the total amount of glycans attached to the glycosylation sites the antibody in a composition.

Embodiment 31. The antibody according to Embodiment 29, wherein the antibody has a glycosylation pattern having one or more of the following characteristics
a relative amount of glycans carrying a bisecting GlcNAc residue of at least 0.5% of the total amount of glycans attached to the glycosylation sites of the antibody in a composition;
a relative amount of glycans carrying at least one galactose residue of at least 30% of the total amount of glycans attached to the glycosylation sites of the antibody in a composition;
a relative amount of glycans carrying a core fucose residue of 40% or less of the total amount of glycans attached to the glycosylation sites of the antibody in a composition.

Embodiment 32. The antibody according to any one of Embodiments 1 to 31, comprising a further agent conjugated thereto.

Embodiment 33. The antibody according to Embodiment 32, wherein the further agent is a chemotherapeutic agent which is coupled to the antibody.

Embodiment 34. The antibody according to Embodiment 33, wherein the chemotherapeutic agent is selected from the group consisting of a maytansinoid, a DNA damaging agent, a DNA alkylating agent and a DNA minor groove binder.

Embodiment 35. The antibody according to Embodiment 33, wherein the chemotherapeutic agent is selected from the group consisting of maytansinol, N^{2′}-deacetyl-N^{2′}-(3-mercapto-1-oxopropyl)-maytansine (DM1), N^{2′}-deacetyl-N^{2′}-(4-mercapto-1-oxopentyl)-maytansine (DM3), and N^{2′}-deacetyl-N^{2′}-(4-methyl-4-mercapto-1-oxopentyl)-maytansine (DM4).

Embodiment 36. The antibody according to Embodiment 33, wherein the chemotherapeutic agent is selected from the group consisting of monomethyl auristatin F (MMAF), monomethyl auristatin E (MMAE) and auristatin T.

Embodiment 37. The antibody according to Embodiment 33, wherein the chemotherapeutic agent is selected from the group consisting of pyrrolobenzodiazepine (PBD), pyrrolobenzodiazepine dimer (PBD dimer), duocarmycin, duocarmycin-hydroxybenzamide-azaindole (DUBA), seco-duocarmycin-hydroxybenzamide-azaindole (seco-DUBA) and doxorubicin.

Embodiment 38. The antibody according to Embodiment 33, wherein the chemotherapeutic agent is selected from the group consisting of indolinobenzodiazepine and oxazolidinobenzodiazepine.

Embodiment 39. The antibody according to Embodiment 33, wherein the chemotherapeutic agent is calicheamicin.

Embodiment 40. The antibody according to Embodiment 32, wherein the further agent is a polypeptide or protein which is fused to a polypeptide chain of the antibody.

Embodiment 41. The antibody according to Embodiment 40, wherein the antibody comprises two antibody heavy chains and two antibody light chains and a further agent being a polypeptide or protein is fused to each of the C termini of said antibody heavy chains or to each of the C termini of said antibody light chains.

Embodiment 42. The antibody according to Embodiment 40 or 41, wherein the further agent is selected from the group consisting of cytokines, chemokines, other antibodies, antigen binding fragments, enzymes and binding domains.

Embodiment 43. The antibody according to Embodiment 41, wherein the further agent is a scFv fragment specifically binding to CD3, and one of said further agent is fused to the C terminus of each antibody heavy chain.

Embodiment 44. The antibody according to Embodiment 41, wherein the further agent is a scFv fragment specifically binding to PDL1, and one of said further agent is fused to the C terminus of each antibody light chain.

Embodiment 45. A nucleic acid encoding the antibody according to any one of Embodiments 1 to 44.

Embodiment 46. An expression cassette or vector comprising the nucleic acid according to Embodiment 75 and a promoter operatively connected with said nucleic acid.

Embodiment 47. A host cell comprising the nucleic acid according to Embodiment 45 or the expression cassette or vector according to Embodiment 46.

Embodiment 48. A pharmaceutical composition comprising the antibody or conjugate according to any one of Embodiments 1 to 44 and one or more further components selected from the group consisting of solvents, diluents, and excipients.

Embodiment 49. The antibody according to any one of Embodiments 1 to 44 or the pharmaceutical composition according to Embodiment 48 for use in medicine.

Embodiment 50. The antibody according to any one of Embodiments 1 to 44 or the pharmaceutical composition according to Embodiment 48 for use in the treatment, prognosis, diagnosis, detecting and/or monitoring of diseases associated with abnormal cell growth such as cancer; infections such as bacterial, viral, fungal or parasitic infections; inflammatory diseases such as autoimmune diseases and inflammatory bowel diseases; and diseases associated with a reduce immune activity such as immunodeficiencies.

Embodiment 51. The antibody or pharmaceutical composition according to Embodiment 50 for use in the treatment of cancer, in particular the cancer expressing TA-MUC1, wherein the cancer is selected from the group consisting of cancer of the ovarian cancer, breast cancer, pancreatic cancer, lung cancer, colon cancer, stomach cancer, liver cancer, kidney cancer, blood cancer, endometrial cancer, thyroid cancer, leukemia, seminomas, melanomas, carcinomas, teratomas, lymphomas, sarcomas, mesotheliomas, neuroblastomas, gliomas, rectal cancer, adrenal cancer, skin cancer, cancer of the brain, cervical cancer, intestinal cancer, intestine cancer, head and neck cancer, gastrointestinal cancer, lymph node cancer, esophagus cancer, colorectal cancer, ear, nose and throat (ENT) cancer, prostate cancer, bladder cancer, cancer of the uterus and the metastases thereof.

Embodiment 52. The antibody or pharmaceutical composition according to Embodiment 50 for use in the treatment of infections, wherein the infection is selected from the group consisting of bacterial infections, viral infections, fungal infections and parasitic infections.

Embodiment 53. The antibody or pharmaceutical composition according to Embodiment 50 for use in the treatment of autoimmune diseases, wherein the autoimmune disease is selected from the group consisting of celiac disease, diabetes mellitus type 1, Graves disease, inflammatory bowel disease, multiple sclerosis, psoriasis, rheumatoid arthritis and systemic lupus erythematosus.

Embodiment 54. A method of increasing the MUC1 binding affinity of an antibody comprising
i. a heavy chain variable region, which
   a) has an amino acid sequence which is at least 90% identical to the amino acid sequence of SEQ ID NO: 11, and
   b) comprises the complementarity-determining regions (CDRs) CDR-H1 having the amino acid sequence of SEQ ID NO: 1, CDR-H2 having the amino acid sequence of SEQ ID NO: 8 and CDR-H3 having the amino acid sequence of SEQ ID NO: 3, and
ii. a light chain variable region, which
   a) has an amino acid sequence which is at least 90% identical to the amino acid sequence of SEQ ID NO: 12, and
   b) comprises the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6;
      the method comprising the step of substituting the amino acid residue at position 8 of CDR-H2 with any amino acid residue except asparagine, resulting in CDR-H2 having the amino acid sequence of SEQ ID NO: 2.

Embodiment 55. A method of increasing the MUC1 binding affinity of an antibody comprising
i. a heavy chain variable region, which
   a) has an amino acid sequence which is at least 95% identical to the amino acid sequence of SEQ ID NO: 11, and
   b) comprises the complementarity-determining regions (CDRs) CDR-H1 having the amino acid sequence of SEQ ID NO: 1, CDR-H2 having the amino acid sequence of SEQ ID NO: 8 and CDR-H3 having the amino acid sequence of SEQ ID NO: 3, and
ii. a light chain variable region, which
   a) has an amino acid sequence which is at least 95% identical to the amino acid sequence of SEQ ID NO: 12, and
   b) comprises the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6; the method comprising the step of substituting the amino acid residue at position 8 of CDR-H2 with any amino acid residue except asparagine, resulting in CDR-H2 having the amino acid sequence of SEQ ID NO: 2.

Embodiment 56. The method according to embodiment 54 or 55, wherein substituting the amino acid residue at position 8 of CDR-H2 is achieved by introducing a mutation into the nucleic acid coding for the antibody, wherein the mutation is introduced in the codon coding for said amino acid residue.

Embodiment 57. The method according to any one of Embodiments 54 to 56, comprising the steps of
a) providing a nucleic acid coding for the antibody which MUC1 binding affinity is to be increased;
b) introducing a mutation into said nucleic acid to produce a mutated nucleic acid, wherein the mutation is introduced in the codon coding for the amino acid residue at position 8 of CDR-H2 so that said codon codes for any amino acid residue except asparagine; and
c) expressing the mutated nucleic acid to produce an antibody with increased MUC1 binding affinity.

Embodiment 58. A method of producing an antibody with increased MUC1 binding affinity, comprising
a) providing a nucleic acid coding for an antibody which comprises
i. a heavy chain variable region, which
   i1. has an amino acid sequence which is at least 90% identical to the amino acid sequence of SEQ ID NO: 11, and
   i2. comprises the complementarity-determining regions (CDRs) CDR-H1 having the amino acid sequence of SEQ ID NO: 1, CDR-H2 having the amino acid sequence of SEQ ID NO: 8 and CDR-H3 having the amino acid sequence of SEQ ID NO: 3, and
ii. a light chain variable region, which
   ii1. has an amino acid sequence which is at least 90% identical to the amino acid sequence of SEQ ID NO: 12, and
   ii2. comprises the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6;
b) introducing a mutation into said nucleic acid to produce a mutated nucleic acid, wherein the mutation is introduced in the codon coding for the amino acid residue at position 8 of CDR-H2 so that said codon codes for any amino acid residue except asparagine; and
c) producing the antibody with increased MUC1 binding affinity by expressing the mutated nucleic acid in a host cell.

Embodiment 59. A method of producing an antibody with increased MUC1 binding affinity, comprising
a) providing a nucleic acid coding for an antibody which comprises
i) a heavy chain variable region, which
i1) has an amino acid sequence which is at least 95% identical to the amino acid sequence of SEQ ID NO: 11, and
i2) comprises the complementarity-determining regions (CDRs) CDR-H1 having the amino acid sequence of SEQ ID NO: 1, CDR-H2 having the amino acid sequence of SEQ ID NO: 8 and CDR-H3 having the amino acid sequence of SEQ ID NO: 3, and
ii) a light chain variable region, which
ii1) has an amino acid sequence which is at least 95% identical to the amino acid sequence of SEQ ID NO: 12, and
ii2) comprises the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6;
b) introducing a mutation into said nucleic acid to produce a mutated nucleic acid, wherein the mutation is introduced in the codon coding for the amino acid residue at position 8 of CDR-H2 so that said codon codes for any amino acid residue except asparagine; and
c) producing the antibody with increased MUC1 binding affinity by expressing the mutated nucleic acid in a host cell.

Embodiment 60. The method according to any one of Embodiments 54 to 59, wherein the antibody comprises two heavy chains, each comprising the heavy chain variable region, a CH1 domain, a hinge region, a CH2 domain and a CH3 domain.

Embodiment 61. The method according to Embodiment 60, wherein the antibody is an IgG-type antibody, in particular an IgG1, IgG2 or IgG4-type antibody.

Embodiment 62. The method according to any one of Embodiments 54 to 60, wherein the antibody comprises two light chains, each comprising the light chain variable region and a CL domain.

Embodiment 63. The method according to Embodiment 62, wherein the light chain is a κ-type light chain.

Embodiment 64. The method according to any one of Embodiments 54 to 63, wherein the antibody with increased MUC1 binding affinity is an antibody as defined in any one of Embodiments 1 to 44.

Embodiment 65. A method for treating cancer in a subject in need thereof comprising, administering to the subject with cancer, in particular cancer expressing TA-MUC1, a therapeutically effective amount of the antibody according to any one of Embodiments 1 to 44 or the composition according to Embodiment 48.

Embodiment 66. The method for treating cancer according to Embodiment 65, wherein the cancer is selected from the group consisting of ovarian cancer, breast cancer, pancreatic cancer, lung cancer, colon cancer, stomach cancer, liver cancer, kidney cancer, blood cancer, endometrial cancer, thyroid cancer, leukemia, seminomas, melanomas, carcinomas, teratomas, lymphomas, sarcomas, mesotheliomas, neuroblastomas, gliomas, rectal cancer, adrenal cancer, skin cancer, cancer of the brain, cervical cancer, intestinal cancer, intestine cancer, head and neck cancer, gastrointestinal cancer, lymph node cancer, esophagus cancer, colorectal cancer, ear, nose and throat (ENT) cancer, prostate cancer, bladder cancer, cancer of the uterus and the metastases thereof.

### FIGURES

FIG. 1 shows ELISA binding curves of the anti-MUC1 antibodies to different MUC1 peptides. (A) shows antigen binding of PankoMab N54Q (PM-N54Q) lacking Fab glycosylation and PankoMab comprising Fab glycosylation (PM) to the MUC1 peptide comprising the epitope sequence PDTR (SEQ ID NO: 13). The threonine of the MUC1 peptide is glycosylated with Tn, sTn, TF or sTF. (B) shows binding of PankoMab and PankoMab N54Q to the MUC1 peptide comprising the epitope sequence variant PESR (SEQ ID NO: 14). The serine of the MUC1 peptide is glycosylated with Tn. (C) shows binding of PankoMab N54Q to the MUC1 peptide comprising the epitope sequence PDTR. The threonine of the MUC1 peptide is glycosylated with Tn or not glycosylated. (D) shows binding of several N54X variants to Tn-PDTR (SEQ ID NO: 13) MUC1 peptide compared to PankoMab comprising Fab glycosylation diluted from cell culture supernatant of transiently transfected cells. (E) shows binding curves of three purified N54X variants without Fab glycosylation in comparison to PankoMab with Fab glycosylation on Tn-PDTR (SEQ ID NO: 13), TF-PDTR (SEQ ID NO: 13) and non-glycosylated PDTR (SEQ ID NO: 13). MUC1 peptide. (F) shows binding of two framework variants of PM-N54Q to Tn-PDTR (SEQ ID NO: 13) MUC1 peptide compared to PankoMab with Fab glycosylation. For framework variant mf-a nine amino acids are mutated in the VH and three in the VL framework, for mf-b also nine amino acids are mutated in the VH and four in the VL framework.

FIG. 2 shows surface plasmon resonance (Biacore) binding of the anti-MUC1 antibodies PM and PM-N54Q to a Tn-glycosylated PDTR (SEQ ID NO: 13)-MUC1 peptide. The maximal binding signal of different concentrations of PankoMab N54Q and PankoMab are plotted against the antibody concentration.

FIG. 3 shows results of Fluorescence Proximity Sensing on DRX instrument. Association and dissociation curves are shown. (A) PM with Fab glycosylation compared to (B) PM-N54Q without Fab glycosylation.

FIG. 4 shows an SDS acrylamide gel of an electrophoretic separation of PankoMab N54Q and PankoMab under non-reducing (left) and reducing (right) conditions. Lane 1: PankoMab N54Q after capture step; lane 2: PankoMab N54Q after polishing step; lane 3: PankoMab after capture step; lane 4: PankoMab after polishing step; lane 5: molecular weight marker.

FIG. 5 shows the Coomassie blue stained gel of an isoelectric focusing assay with PankoMab N54Q lacking Fab glycosylation and PankoMab being Fab-glycosylated. Lane 1: PankoMab with Fab glycosylation; lane 2: PankoMab N54Q without Fab glycosylation.

FIG. 6 shows anti-MUC1 antibody binding to Fcγ receptor IIIa. Increasing concentrations of the antibody PankoMab N54Q or PankoMab displace rabbit-anti-mouse coupled acceptor beads from FcγRIIIa loaded donor beads, thereby reducing the detected chemiluminescence. In FIG. 6A low-fucosylated antibodies and in FIG. 6B high-fucosylated antibodies were applied into the assay.

FIG. 7 shows binding of the anti-MUC1 antibodies PM-N54Q, PM-N54D and PM with Fab glycosylation to the tumor cell lines (A) CaOV-3 and (B) HSC-4 as analyzed by flow cytometry.

FIG. 8 shows the amino acid sequence of the heavy chain of the humanized antibody PankoMab N54Q (SEQ ID No: 15, wherein the amino acid at position 57 is Gln, namely SEQ ID No: 22).

FIG. 9 shows the amino acid sequence of the light chain of the humanized antibody PankoMab N54Q (SEQ ID No: 16).

FIG. 10 shows the amino acid sequence of the heavy chain of the humanized antibody PankoMab (SEQ ID No: 19).

FIG. 11 shows the amino acid sequence of the heavy chain of chimeric antibody PankoMab N54Q (SEQ ID No: 20, wherein the amino acid at position 76 is Gln, namely SEQ ID No: 23).

FIG. 12 shows the amino acid sequence of the light chain of chimeric antibody PankoMab N54Q (SEQ ID No: 21)

### EXAMPLES

### Example 1: Production of Anti-MUC1 Antibodies

The nucleic acid sequence of the heavy chain of humanized PankoMab antibody (see, e.g., WO 2011/012309) was modified by mutating the codon for Asn54 according to the Kabat/EU numbering system (amino acid position 57 in SEQ ID NO: 11) into the codon for any amino acid except Asn, especially for Gln.

### 1) Production of the Anti-MUC1 Antibodies in a Human Myeloid Leukemia Derived Cell Line

Vectors comprising the coding sequences of the γ1-type heavy chain and the κ-type light chain of the mutated antibodies were transfected into the human myeloid leukemia derived cell line NM-H9D8 (DSM ACC2806). The different aMUC1-antibodies comprising the N54X mutation (PankoMab N54X/PM-N54X whereby X is any amino acid except N/Asn) or amino acid mutations in the framework sequences of the VH and VL were expressed in the obtained clones, producing the constructs with a human glycosylation pattern. The concentration of the αMUC1-antibodies in the supernatant was determined by Octet measurement using Protein A coated pins or were quantified by UV280 absorbance after purification by protein A chromatography. The binding characteristics of the different αMUC1-antibodies were determined by Antigen-ELISA (see example 2), and selected purified antibodies were also analyzed by Scatchard analysis (see example 3), by Biacore (see example 4a), by DRX² switchSENSE® technology (see example 4b), or by flow cytometry (example 7).

In addition, PM-N54Q and non-mutated PankoMab with Fab-glycosylation were also expressed in the human myeloid leukemia derived cell line NM-H9D8-E6Q12 (DSM ACC2856) expressing antibody with reduced fucose. Together with the same antibodies expressed in NM-H9D8, these antibodies were purified and analyzed in example 6 for their binding behavior to Fc gamma receptor III A.

### 2) Production of the Anti-MUC1 Antibody in CHO Cell Line

PM-N54Q encoding sequences (nucleotide sequence of heavy chain of PM-N54Q represented by SEQ ID NO: 17 and nucleotide sequence of light chain of PM-N54Q represented by SEQ ID NO: 18) which was synthesized by GeneArt™ of ThermoFisher scientific were cloned into expression vectors and resulting plasmids were electro-transfected into CHO cells. Pooled cells grown under selection pressure were applied to manufacture PM-N54Q mutant antibody with general procedures.

### Example 2: Antigen ELISA

The antigen binding characteristics of PankoMab N54X, wherein the N-glycosylation site in the Fab part is knocked out, was compared to PankoMab having an N-glycosylation site in its Fab part.

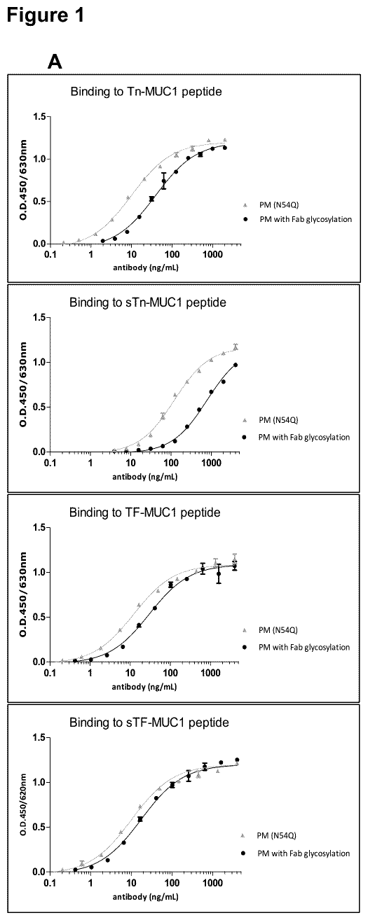

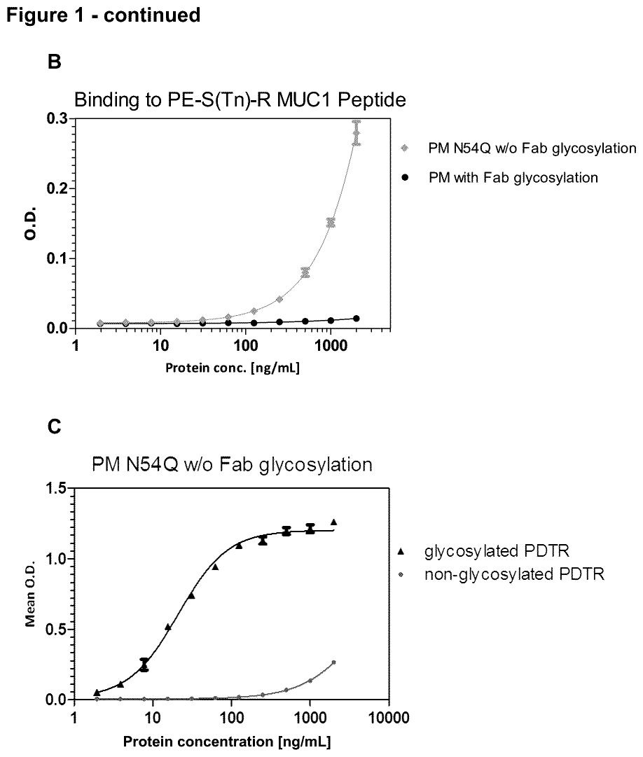

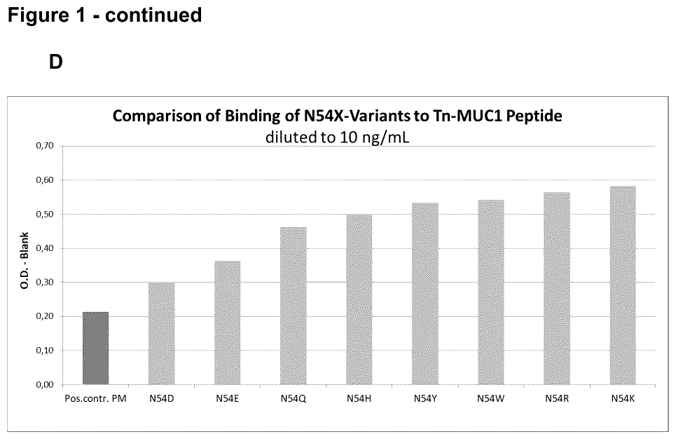

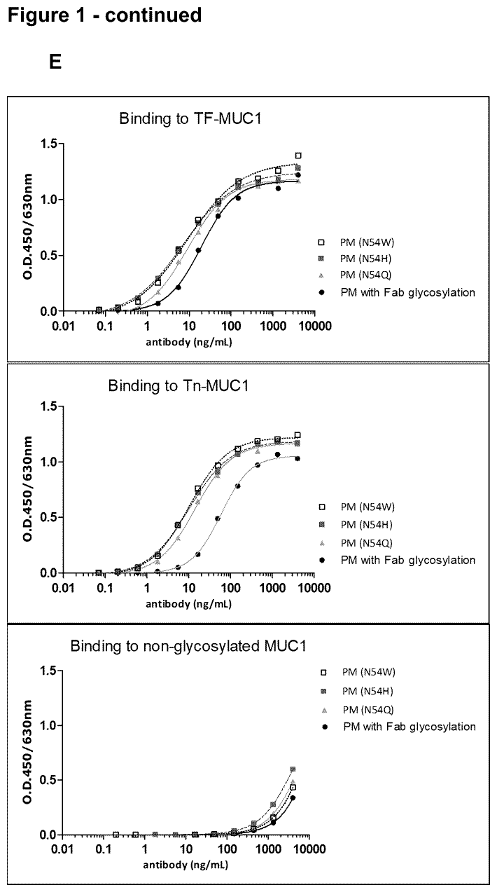

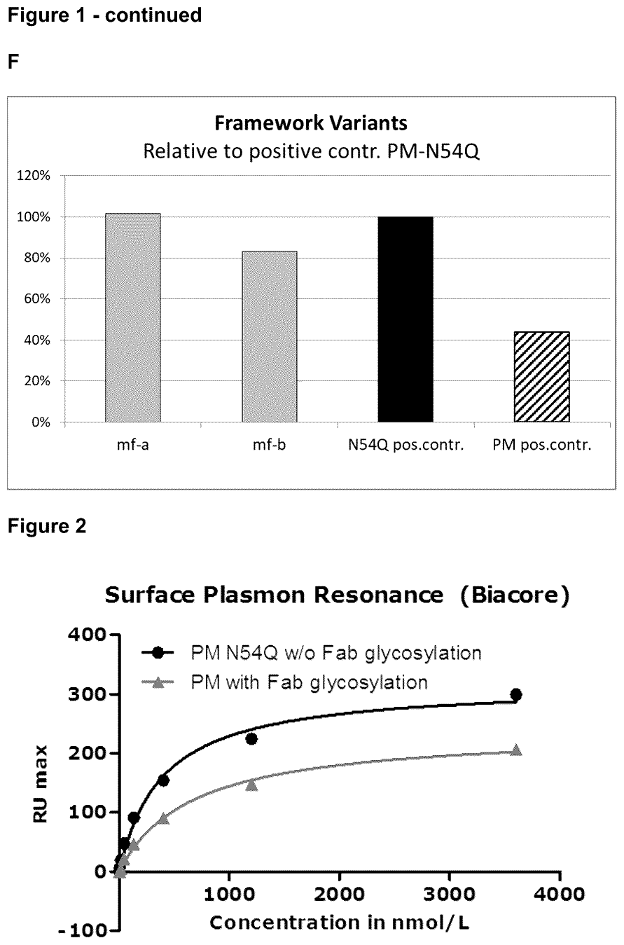

Binding characteristics of the Fab-deglycosylated version of the MUC1-specific antibody PankoMab (PM-N54Q) compared to the (glycosylated) PankoMab-GEX® were analyzed using differently glycosylated and the non-glycosylated MUC1-derived tandem repeat peptides in ELISA studies. In principle, both antibodies show the same gradation by means of binding to glycosylated PDTR (SEQ ID NO: 13) peptides (APPAHGVTSAPD-T(X)-RPAPGSTAPPAHGVTSA) (SEQ ID NO: 24) with different glycosylations at T: Strongest binding was observed to the PDTR peptide carrying a Galβ1-3GaNAcₐₗₚₕₐ (TF) followed by sialylated TF and GalNAcₐₗₚₕₐ (Tn) O-glycosylation. Binding to sialylated GalNAcₐₗₚₕₐ (sTn) O-glycosylation was significantly lower. As PankoMab-GEX®, PM-N54Q showed only little binding activity to non-glycosylated MUC1 PDTR (SEQ ID NO: 13) peptide indicating adequate tumor specificity (FIG. 1C).

However, in comparison to PankoMab-GEX® four-fold higher binding was found for PM-N54Q in the TA-MUC1 antigen ELISA using the biotinylated glycopeptide carrying a GalNAcₐₗₚₕₐ (Tn) O-glycan. PM-N54Q binds about seven-fold better to the same MUC1 peptide when glycosylated with sialylated GalNAcₐₗₚₕₐ (sTn). The binding to GalI1-3GalNAcₐₗₚₕₐ (TF) and sialylated TF (sTF) at the threonine of the PDTR (SEQ ID NO: 13)-sequence (FIG. 1A) was two-fold better for PM-N54Q.

Both antibodies show strongly diminished binding to the MUC1 peptide variant APPAHGVTSAPE-S(Tn)-RPAPGSTAPPAHGVTSA (SEQ ID NO: 25) with Tn glycosylation at the serine compared to that at PDT(Tn)R-peptide (SEQ ID NO: 13). However, also here the Fab-deglycosylated PM-N54Q binds significantly stronger than PankoMab-GEX® (FIG. 1B).

Different other Fab-deglycosylated PM-N54X variants were compared to PankoMab having an N-glycosylation in its Fab part. First, all variants were compared directly from the supernatant, without purification. The concentration was determined by Octet. All PM-N54X variants bound better than Fab-glycosylated PM. In addition, a clear trend depending on the chemical properties of the amino acid side chain was visible. Carboxylic acid groups at the side chain showed the lowest binding enhancement. Best binding was observed for amino acids with one or two nitrogens (as primary or secondary amines) (FIG. 1D).

In addition, selected Fab-deglycosylated variants (PM-N54H, -W and -Q) were purified by Protein A chromatography and analyzed on ELISA (FIG. 1E). The improvement of binding to TF-MUC1 peptide is about 5- to 8-fold and to Tn-MUC1 peptide about 2- to 3-fold compared to PankoMab with Fab-glycosylation, respectively.

Furthermore, two different framework variants of the PM-N54Q were analyzed for the binding to the Tn-glycosylated PDTR-(SEQ ID NO: 13) MUC1 peptide in ELISA (see FIG. 1F). The framework variant mf-a carries nine amino acid mutations in the VH and three in the VL framework; the variant mf-b carries also nine amino acid mutations in the VH and four in the VL framework. Both mutated variants show similar binding compared to the PM-N54Q antibody.

### Example 3: Saturation Binding Analyses of Anti-MUC1 Antibodies to MCF-7 and ZR-75-1 Cells

Two factors are especially critical for the therapeutic suitability of an antibody: the affinity and number of binding sites of an antibody on tumor cells.

Binding of the Fab-deglycosylated version of the MUC1-specific antibody PankoMab (PM-N54Q) on TA-MUC-1 positive human tumor cell lines was evaluated using radiolabeled antibodies by saturated binding analysis on the human mamma carcinoma cell lines ZR-75-1 and MCF-7 in comparison to Fab-glycosylated PankoMab-GEX®. The antibodies were chelated with a 12-fold molar excess of p-SCN-Benzyl-DTPA in 50 mM sodium carbonate, 150 mM NaCl, pH 8.7, for 2 h at 37° C., followed by over-night incubation at 2-8° C. Free chelator was removed over desalting columns and dead-end filtration (50 kDa cut-off, 6× buffer exchange to PBS). The chelated antibodies were radiolabeled with carrier-free ¹¹¹In (2 μCi/μg antibody) for 1 h at 37° C. in 6 mM phosphate, 1.6 mM KCl, 80 mM NaCl, 0.2 M Na-acetate, 0.1 M HCl. The preparations were neutralized by addition of 8-9 fold volume of 10× concentrated PBS. About 1/50 volume of fetal bovine serum were added to the neutralized labelled antibody preparation. Per cell binding approach 1*10⁶ cells were used. Several concentrations of labelled antibodies were added to the pelleted cells (30-1000 ng/200 μL in 1% BSA/PBS). The resuspended cell-antibody mixtures were measured in a gamma-counter and incubated 1 h at 4° C. Cells with bound antibodies were separated by centrifugation and washed with 1% BSA/PBS for another hour at 4° C. The cell pellet was then measured for bound ¹¹¹In-labelled antibody in a gamma counter. Evaluation was performed by “one-site specific ka” in GraphPad Prism. The obtained data are summarized in Table 1. The data show the high affinity and very high number of binding sites of PM-N54Q on these tumor cells. The binding was more than 2.5-fold higher than for PankoMab-GEX® and also the number of binding sites was slightly increased.

TABLE 1
Association constant and antigen
binding sites on MUC1⁺ tumor cells

| | ZR-75-1 | MCF-7 |
|---|---|---|
| Kₐₛₛ [1/M] | | |
| PM w Fab glyc. | 1.2 × 10⁷ | 3 × 10⁷ |
| PM N54Q w/o Fab glyc. | 3.4 × 10⁷ | 7.8 × 10⁷ |
| Binding sites | | |
| PM w Fab glyc. | 20 × 10⁵ | 0.6 × 10⁵ |
| PM N54Q w/o Fab glyc. | 30 × 10⁵ | 0.9 × 10⁵ |

### Example 4a: Surface Plasmon Resonance (BiaCore) Analysis

Binding of the Fab-deglycosylated version of the MUC1-specific antibody PankoMab (PM-N54Q) on TA-MUC-1 derived glycosylated peptide was evaluated by surface plasmon resonance analysis (Biacore). A streptavidin sensor chip was coated with biotinylated TA-MUC1 peptide (Tn glycosylated or not glycosylated). PankoMab and PM-N54Q were diluted sequentially 1:3 from 3,600 to 4.9 nM in HPS-EP. The dilutions were injected at 50 μL/min. Maximal binding of each concentration was determined as response units (RU), respectively, and evaluated with GraphPad Prism using “one-site specific binding”. FIG. 2 shows the obtained binding curves with PM-N54Q compared to PankoMab-GEX®. Affinities (K_{D}) of 388 nM and 652 nM were calculated for PM-N54Q and PankoMab-GEX®, respectively. Therefore, in this experimental setting a nearly two-fold increase in affinity was detectable.

### Example 4b: Fluorescence Proximity Sensing (by DRX², Dynamic Biosensors)

A new method to determine binding constants and affinity is the fluorescence proximity sensing using single stranded DNA (96mer) spotted on a chip and complementary DNA coupled to a ligand. In the present study streptavidin was used as a ligand to capture biotinylated TA-MUC1 peptides. Binding of PankoMab to the peptides resulted in a fluorescence change. On- and off-rates can be calculated during association and dissociation. Due to a higher sensitivity faster interactions can be monitored compared to surface plasmon resonance. This results in binding kinetics different from SPR but more comparable to the “gold standard” method KinExA, measured in a liquid system.

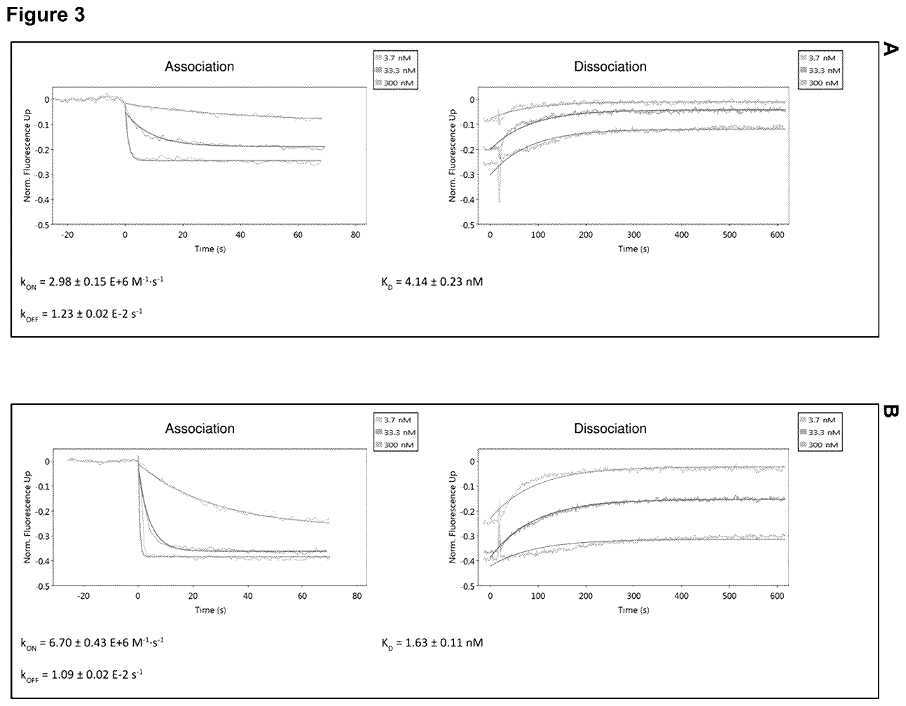

PankoMab and PM-N54Q were diluted from 300 nM in 1:9 steps to 3.67 nM in PE140 buffer and applied to the chip-bound peptides. Binding curves were evaluated by mono-exponential global fit (instrument software). Binding curves of PM and PM-N54Q are exemplarily shown in FIGS. 3A and B. Calculated affinities of PankoMab variants are shown in Table 2:

TABLE 2
Dissociation constants of PankoMab
variants to antigen peptide

| PankoMab variant | K_{D} |
|---|---|
| PM with Fab glycosylation | 4.1 nM |
| PM-N54D | 1.9 nM |
| PM-N54Q | 1.6 nM |
| PM-N54H | 0.6 nM |

### Example 5: Biochemical Characterization

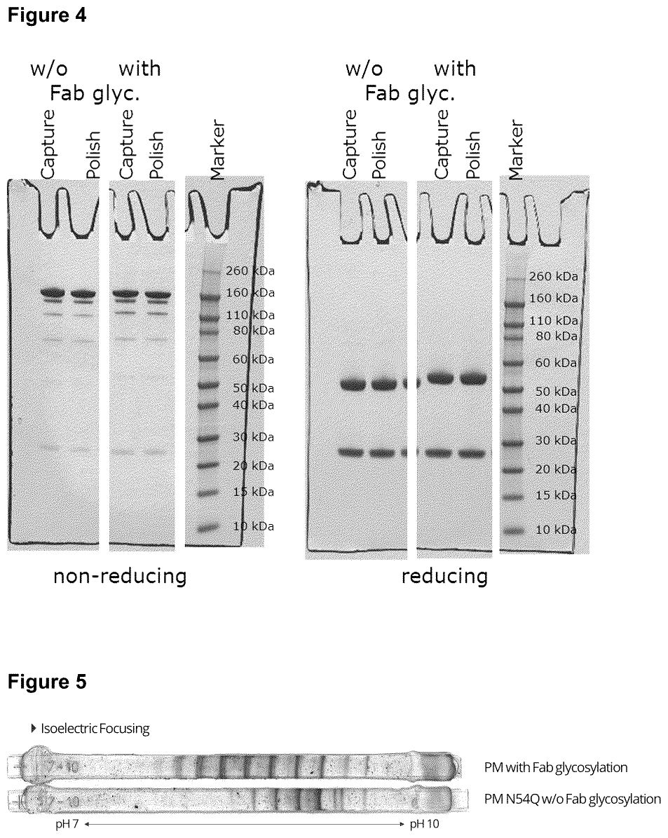

Non-reducing and reducing SDS-PAGE is used to analyze purity and identity of an antibody. The band pattern in non-reducing gels shows the major band at about 160 kDa and methodical artefacts of heavy and light chains and combinations thereof (˜25, 50-55, 75, 110, 135 kDa). Reducing gels show distinct light and heavy chain bands at 25 and 50-55 kDa. Due to lack of the Fab glycosylation PM-N54Q has a smaller heavy chain, as expected (see FIG. 4, right).

The charge profile is clearly different, as shown by isoelectric focusing (IEF; see FIG. 5). The Fab glycosylation is considerably sialylated, whereas the Fc glycosylation is only minimally sialylated. Thus PankoMab-GEX® has more charged isoforms than PM-N54Q, reflecting its higher level of negatively charged sialic acids in the Fab part.

### Example 6: Fcγ Receptor Binding

FcγR binding assays for FcγRIIIa (CD16a) are based on the AlphaScreen® technology of PerkinElmer. The AlphaScreen® platform relies on simple bead-based technology of PerkinElmer and is a more efficient alternative to traditional ELISA since no washing steps are necessary.

For the receptor binding assays, His-tagged FcγRIIIa (Glycotope GmbH) is captured by Ni-chelate donor beads. Anti-MUC1 antibodies and rabbit-anti-mouse coupled acceptor beads compete for binding to FcγR. In case of interaction of FcγR with rabbit-anti-mouse-bound acceptor beads, donor and acceptor beads come into close proximity which leads, upon laser excitation at 680 nm, to light emission. A maximum signal is achieved (signalₘₐₓ) without a competitor. In case of competition, where a test antibody binds to FcγR, the signalₘₐₓ is reduced in a concentration-dependent manner. Chemiluminescence was quantified by measurement at 520-620 nm (AlphaScreen® method) using an EnSpire 2300 multilabel reader (PerkinElmer). All results were expressed as the mean±standard deviation of duplicate samples. The data were evaluated and calculated using non-linear curve fitting (sigmoidal dose-response variable slope) with GraphPad Prism 5 software. As a result, a concentration dependent sigmoidal curve was obtained, which is defined by top-plateau, bottom-plateau, slope and EC₅₀.

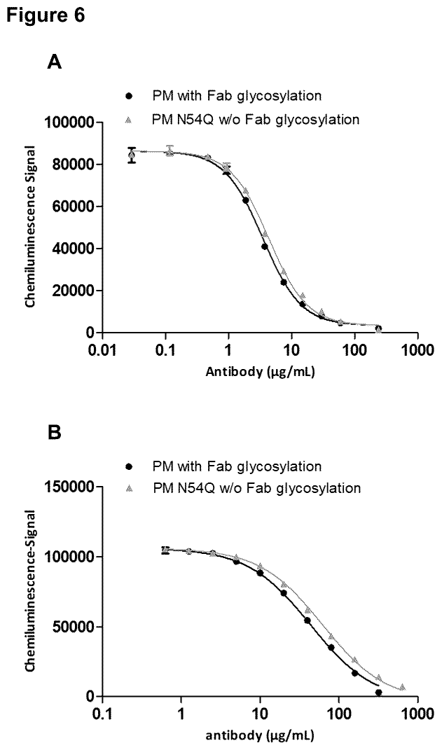

As shown in FIGS. 6A and B, the FcγRIIIa binding affinity was comparable for PankoMab N54Q and PankoMab whereby in Figure A low-fucosylated antibodies and in Figure B high-fucosylated antibodies were applied into the assay. Hence, removal of the Fab glycosylation did not affect receptor interaction of the antibody.

### Example 7: Binding to Cellular TA-MUC1

N54Q and N54D were transiently expressed and purified by protein A chromatography. Binding of the two variants to cell surface TA-MUC1 was compared to PM with Fab glycosylation using two different carcinoma cell lines. The tongue squamous cell carcinoma line HSC-4 expresses TA-MUC1 to a medium degree and the ovarian carcinoma cell line CaOV-3 to a high degree. Tumor cells were incubated with antibodies in serial dilutions and bound antibodies were detected using a Phycoerythrin-conjugated goat anti-human IgG (heavy and light chain) antibody. A human IgG control was included to control for background staining. Binding was analyzed by flow cytometry.

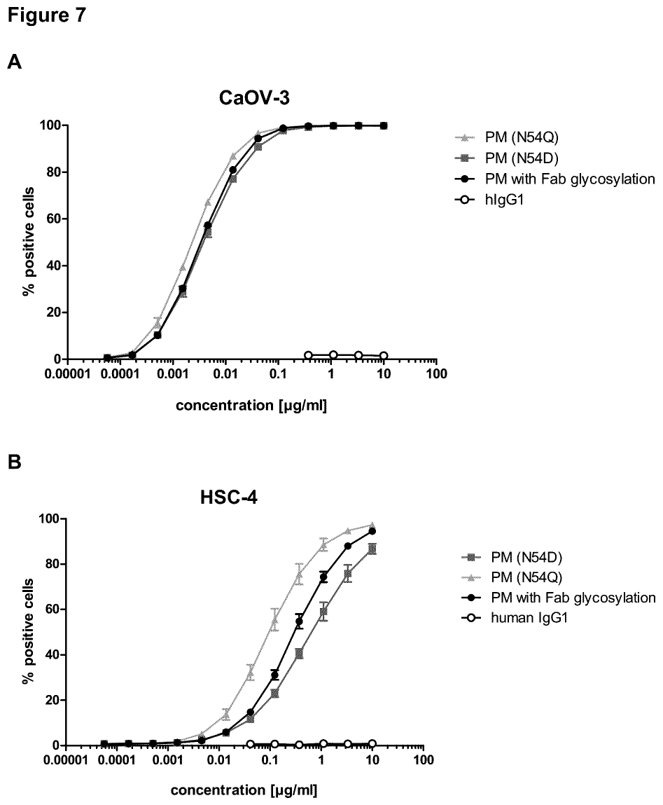

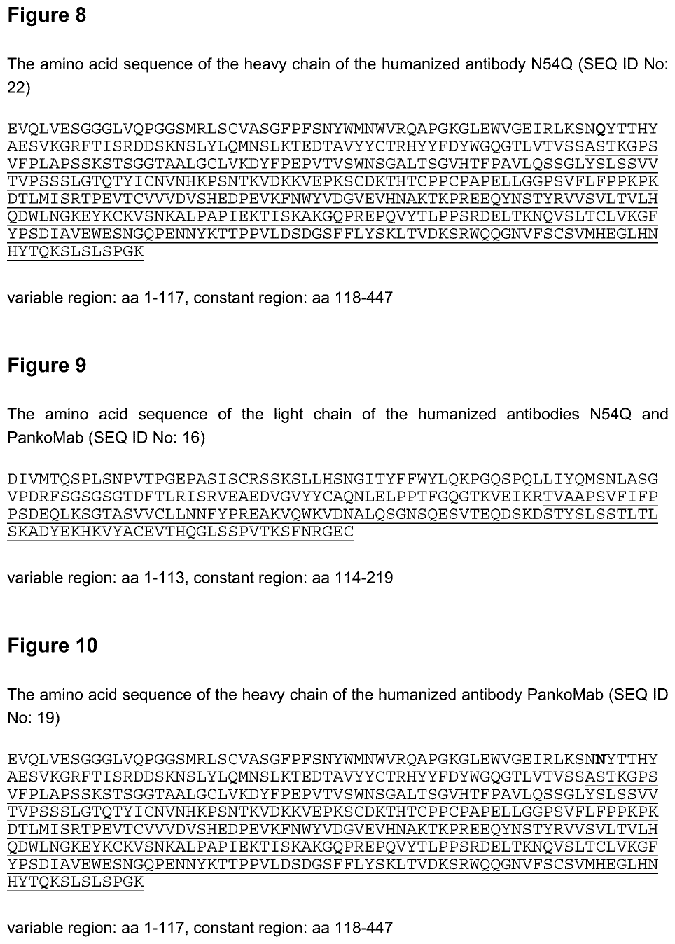

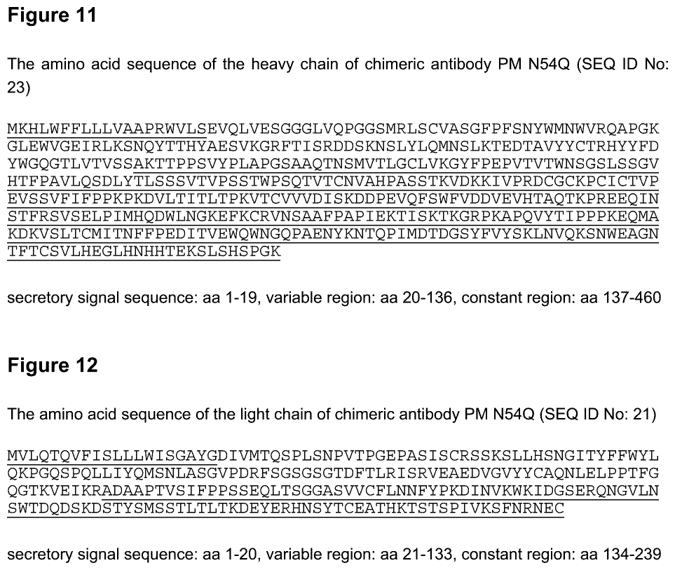

The analyzed constructs PM, PM-N54Q and PM-N54D show strong and specific binding to the TA-MUC1 expressing HSC-4 and CaOV-3 cells compared to a human IgG1 control (FIG. 7). The binding of PM-N54D to the TA-MUC1^{high} CaOV-3 cells was comparable to PM with Fab glycosylation while PM-N54Q showed a slightly better binding (FIG. 7A). Using HSC-4 carcinoma cells that express TA-MUC1 at an intermediate level, the variant PM-N54Q was clearly superior in binding to cellular TA-MUC1 compared to PM while PM-N54D showed an inferior binding compared to PM with Fab glycosylation (FIG. 7B).

### Identification of the Deposited Biological Material

The cell lines DSM ACC 2806, DSM ACC 2807 and DSM ACC 2856 were deposited at the DSMZ—Deutsche Sammlung von Mikroorganismen und Zellkulturen GmbH, Inhoffenstraße 7B, 38124 Braunschweig (DE) by Glycotope GmbH, Robert-Rössle-Str. 10, 13125 Berlin (DE) on the dates indicated in the following table.

| Name of the | Accession | | Date of |
|---|---|---|---|
| Cell Line | Number | Depositor | Deposition |
| NM-H9D8 | DSM ACC 2806 | Glycotope GmbH | Sep. 15, 2006 |
| NM-H9D8-E6 | DSM ACC 2807 | Glycotope GmbH | Oct. 5, 2006 |
| NM-H9D8-E6Q12 | DSM ACC 2856 | Glycotope GmbH | Aug. 8, 2007 |

1. An antibody capable of binding to MUC1, which comprises
(i) a heavy chain variable region comprising the complementarity-determining regions (CDRs) CDR-H1 having the amino acid sequence of SEQ ID NO: 1, CDR-H2 having the amino acid sequence of SEQ ID NO: 2 and CDR-H3 having the amino acid sequence of SEQ ID NO: 3, and
(ii) a light chain variable region comprising the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6
wherein the amino acid at position 8 of SEQ ID NO: 2 is selected from the group consisting of histidine, tryptophan, tyrosine, lysine and arginine.

2. The antibody according to claim 1, wherein the heavy chain variable region has the amino acid sequence of SEQ ID NO: 10 or an amino acid sequence which is at least 90% identical to the amino acid sequence of SEQ ID NO: 10.

3. The antibody according to claim 1, wherein the light chain variable region has the amino acid sequence of SEQ ID NO: 12 or an amino acid sequence which is at least 90% identical to the amino acid sequence of SEQ ID NO: 12.

4. The antibody according to claim 1, wherein the heavy chain variable region of the antibody has the amino acid sequence of SEQ ID NO: 10 and the light chain variable region of the antibody has the amino acid sequence of SEQ ID NO: 12.

5. The antibody according to claim 1, wherein the antibody comprises an Fc region and preferably is an IgG1, IgG2 or IgG4-type antibody.

6. The antibody according to claim 1, wherein the antibody comprises a heavy chain having the amino acid sequence of SEQ ID NO: 15, in particular SEQ ID NO: 22, and a light chain having the amino acid sequence of SEQ ID NO: 16.

7. The antibody according to claim 5, wherein the antibody comprises a glycosylation pattern having one or more of the following characteristics:
(i) a detectable amount of glycans carrying a bisecting GlcNAc residue;
(ii) a relative amount of glycans carrying at least one galactose residue of at least 25% of the total amount of glycans attached to the Fc glycosylation sites of the antibody in a composition.

8. The antibody according to claim 1, wherein the antibody is obtainable by production in a mammalian cell.

9. The antibody according to claim 1, obtainable by production in a human cell line selected from the group consisting of NM-H9D8 (DSM ACC 2806), NM-H9D8-E6 (DSM ACC 2807), NM-H9D8-E6Q12 (DSM ACC 2856) and cell lines derived therefrom.

10. The antibody according to claim 1, wherein the antibody is obtainable by production in a CHO cell line or a cell line derived therefrom.

11. The antibody according to claim 1, wherein the antibody competes for the binding to TA-MUC1 with an antibody comprising a heavy chain variable region having the amino acid sequence of SEQ ID NO: 10 and a light chain variable region having the amino acid sequence of SEQ ID NO: 12.

12. A nucleic acid encoding the antibody according to claim 1.

13. An expression cassette or vector comprising the nucleic acid according to claim 12 and a promoter operatively connected with said nucleic acid.

14. A host cell comprising the nucleic acid according to claim 12.

15. A conjugate comprising the antibody according to claim 1 conjugated to a further agent, wherein the further agent is a polypeptide or protein.

16. The conjugate according to claim 15, wherein the further agent is a cytokine, an immunomodulatory compound, a tumor-specific antibody or an immune checkpoint blocking or activating antibody.

17. A composition comprising the antibody according to claim 1.

18. A method for the diagnosis, detecting and/or monitoring of cancer, an infection, an autoimmune disease or an immunodeficiency disorder, comprising the step of contacting a test sample with the conjugate according to claim 17.

19. The method according to claim 18, wherein the cancer is characterized by expressing TA-MUC1.

20. The method according to claim 18, wherein the cancer is selected from the group consisting of ovarian cancer, breast cancer, pancreatic cancer, lung cancer, colon cancer, stomach cancer, liver cancer, kidney cancer, blood cancer, endometrial cancer, thyroid cancer, leukemias, seminomas, melanomas, carcinomas, teratomas, lymphomas, sarcomas, mesotheliomas, neuroblastomas, gliomas, rectal cancer, adrenal cancer, skin cancer, cancer of the brain, cervical cancer, intestinal cancer, intestine cancer, head and neck cancer, gastrointestinal cancer, lymph node cancer, esophagus cancer, colorectal cancer, ear, nose and throat (ENT) cancer, prostate cancer, bladder cancer, cancer of the uterus and the metastases thereof.

21. A method for the treatment of cancer, an infection, an autoimmune disease or an immunodeficiency disorder, comprising the step of administering the antibody according to claim 1.

22. A method for the diagnosis, detecting and/or monitoring of cancer, an infection, an autoimmune disease or an immunodeficiency disorder, comprising the step of contacting a test sample with the antibody according to claim 1.

23. The method of treatment according to claim 21, wherein the cancer is characterized by expressing TA-MUC1.

24. The method of treatment according to claim 21, wherein the cancer is selected from the group consisting of ovarian cancer, breast cancer, pancreatic cancer, lung cancer, colon cancer, stomach cancer, liver cancer, kidney cancer, blood cancer, endometrial cancer, thyroid cancer, leukemias, seminomas, melanomas, carcinomas, teratomas, lymphomas, sarcomas, mesotheliomas, neuroblastomas, gliomas, rectal cancer, adrenal cancer, skin cancer, cancer of the brain, cervical cancer, intestinal cancer, intestine cancer, head and neck cancer, gastrointestinal cancer, lymph node cancer, esophagus cancer, colorectal cancer, ear, nose and throat (ENT) cancer, prostate cancer, bladder cancer, cancer of the uterus and the metastases thereof.

25. The method of treatment according to claim 21, wherein the antibody is used in combination with a further agent.

26. A method of increasing the MUC1 binding affinity of an antibody comprising
(i) a heavy chain variable region comprising the complementarity-determining regions (CDRs) CDR-H1 having the amino acid sequence of SEQ ID NO: 1, CDR-H2 having the amino acid sequence of SEQ ID NO: 8 and CDR-H3 having the amino acid sequence of SEQ ID NO: 3, and
(ii) a light chain variable region comprising the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6,
the method comprising the step of substituting the amino acid residue at position 8 of CDR-H2 with any amino acid residue except asparagine, resulting in CDR-H2 having the amino acid sequence of SEQ ID NO: 2.

27. The method according to claim 26, wherein substituting the amino acid residue at position 8 of CDR-H2 is achieved by introducing a mutation into the nucleic acid coding for the antibody, wherein the mutation is introduced in the codon coding for said amino acid residue.

28. A method of producing an antibody with increased MUC1 binding affinity, comprising
(a) providing a nucleic acid coding for an antibody which comprises
(i) a heavy chain variable region comprising the complementarity-determining regions (CDRs) CDR-H1 having the amino acid sequence of SEQ ID NO: 1, CDR-H2 having the amino acid sequence of SEQ ID NO: 8 and CDR-H3 having the amino acid sequence of SEQ ID NO: 3, and
(ii) a light chain variable region comprising the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6;
(b) introducing a mutation into said nucleic acid to produce a mutated nucleic acid, wherein the mutation is introduced in the codon coding for the amino acid residue at position 8 of CDR-H2 so that said codon codes for any amino acid residue except asparagine; and
(c) producing the antibody with increased MUC1 binding affinity by expressing the mutated nucleic acid in a host cell.

29. The method according to claim 28, wherein the antibody with increased MUC1 binding affinity comprises
(i) a heavy chain variable region comprising the complementarity-determining regions (CDRs) CDR-H1 having the amino acid sequence of SEQ ID NO: 1, CDR-H2 having the amino acid sequence of SEQ ID NO: 2 and CDR-H3 having the amino acid sequence of SEQ ID NO: 3, and
(ii) a light chain variable region comprising the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6
wherein the amino acid at position 8 of SEQ ID NO: 2 is selected from the group consisting of histidine, tryptophan, tyrosine, lysine and arginine.

30. The method according to claim 22, wherein the cancer is characterized by expressing TA-MUC1.

31. The method according to claim 22, wherein the cancer is selected from the group consisting of ovarian cancer, breast cancer, pancreatic cancer, lung cancer, colon cancer, stomach cancer, liver cancer, kidney cancer, blood cancer, endometrial cancer, thyroid cancer, leukemias, seminomas, melanomas, carcinomas, teratomas, lymphomas, sarcomas, mesotheliomas, neuroblastomas, gliomas, rectal cancer, adrenal cancer, skin cancer, cancer of the brain, cervical cancer, intestinal cancer, intestine cancer, head and neck cancer, gastrointestinal cancer, lymph node cancer, esophagus cancer, colorectal cancer, ear, nose and throat (ENT) cancer, prostate cancer, bladder cancer, cancer of the uterus and the metastases thereof.

32. The method according to claim 22, wherein the antibody is used in combination with a further agent.

33. A method for the treatment of cancer, an infection, an autoimmune disease or an immunodeficiency disorder, comprising the step of administering the conjugate according to claim 15.

34. The method of treatment according to claim 33, wherein the cancer is characterized by expressing TA-MUC1.

35. The method of treatment according to claim 33, wherein the cancer is selected from the group consisting of ovarian cancer, breast cancer, pancreatic cancer, lung cancer, colon cancer, stomach cancer, liver cancer, kidney cancer, blood cancer, endometrial cancer, thyroid cancer, leukemias, seminomas, melanomas, carcinomas, teratomas, lymphomas, sarcomas, mesotheliomas, neuroblastomas, gliomas, rectal cancer, adrenal cancer, skin cancer, cancer of the brain, cervical cancer, intestinal cancer, intestine cancer, head and neck cancer, gastrointestinal cancer, lymph node cancer, esophagus cancer, colorectal cancer, ear, nose and throat (ENT) cancer, prostate cancer, bladder cancer, cancer of the uterus and the metastases thereof.

36. The method of treatment according to claim 33, wherein the conjugate is used in combination with a further agent.

37. The method according to claim 18, wherein the conjugate is used in combination with a further agent.
